(19) United States
(12) Patent Application Publication
(10) Pub. No.: US 2017/0290568 A1
(43) Pub. Date: Oct. 12, 2017

(54) ULTRASOUND DIAGNOSIS APPARATUS AND METHOD OF CONTROLLING THE ULTRASOUND DIAGNOSIS APPARATUS

(71) Applicants: SAMSUNG MEDISON CO., LTD., Hongcheon-gun (KR); SOGANG UNIVERSITY RESEARCH FOUNDATION, Seoul (KR)

(72) Inventors: Jong-sun KO, Hongcheon-gun (KR); Jin-ho CHANG, Seoul (KR); Ju-young MOON, Seoul (KR); Sung-ho KIM, Seoul (KR)

(73) Assignees: SAMSUNG MEDISON CO., LTD., Hongcheon-gun (KR); SOGANG UNIVERSITY RESEARCH FOUNDATION, Seoul (KR)

(21) Appl. No.: 15/481,601

(22) Filed: Apr. 7, 2017

Related U.S. Application Data
(60) Provisional application No. 62/320,037, filed on Apr. 8, 2016.

(30) Foreign Application Priority Data
Jul. 26, 2016 (KR) 10-2016-0094820

(51) Int. Cl. A61B 8/00; A61B 8/14

(52) U.S. Cl. CPC A61B 8/4444; A61B 8/14; A61B 8/461; A61B 8/467; A61B 8/54; A61B 8/4483; A61B 8/4405

(57) ABSTRACT

An ultrasound diagnosis apparatus and a method of controlling the same are provided. The ultrasound diagnosis apparatus includes an ultrasound probe configured to transmit an ultrasound signal to an object and receive an ultrasound echo signal from the object to form a reception signal; a body configured to receive the reception signal from the ultrasound probe and process the reception signal; an impedance matching circuit configured to match an impedance of the ultrasound probe with an impedance of the body; and a controller configured to control the impedance matching circuit to operate as at least one filter that passes or rejects a signal of a specific frequency band, based on the impedance of the ultrasound probe, the impedance of the body, and relevant frequency characteristics of the reception signal. The at least one filter is controlled to match the impedance of the ultrasound probe with the impedance of the body and is controlled so that the reception signal has the relevant frequency characteristics.

### CROSS-REFERENCE TO RELATED APPLICATION

This application claims the benefit of U.S. Provisional Patent Application No. 62/320,037, filed on Apr. 8, 2016, in the U.S. Patent and Trademark Office, and the benefit of Korean Patent Application No. 10-2016-0094820, filed on Jul. 26, 2016, in the Korean Intellectual Property Office, the disclosures of which are incorporated herein in their entireties by reference.

### BACKGROUND

### 1. Field

One or more embodiments relate to an ultrasound diagnosis apparatus and a method of controlling the ultrasound diagnosis apparatus.

### 2. Description of the Related Art

Ultrasound diagnosis apparatuses transmit an ultrasound signal generated by a transducer of a probe to an object and detects information regarding an echo signal reflected from the object, thereby obtaining at least one image of a part (e.g., a soft tissue or a blood stream) inside the object.

However, a difference between the impedance value of a body of an ultrasound diagnosis apparatus and the impedance value of an ultrasound probe exists. Due to this difference between the impedance values of the ultrasound diagnosis apparatus body and the ultrasound probe, a reception signal received by the ultrasound probe is not fully delivered to the ultrasound diagnosis apparatus. In other words, when the reception signal is transmitted from the ultrasound probe to the ultrasound diagnosis apparatus, a portion of the electrical energy of the reception signal is lost. In addition, a reception signal received by the ultrasound diagnosis apparatus body has a reduced frequency band compared to the reception signal received by the ultrasound probe.

One method to address the loss of the electrical energy of the received signal and the reduction of the frequency band of the received signal due to the difference between the impedance values of the ultrasound diagnosis apparatus body and the ultrasound probe is to match the impedance of the ultrasound diagnosis apparatus body with the impedance of the ultrasound probe by using an impedance matching circuit located between the ultrasound diagnosis apparatus body and the ultrasound probe. When the impedance of the ultrasound probe is matched with the impedance of the ultrasound diagnosis apparatus body, loss of the electrical energy of the reception signal received by the ultrasound diagnosis apparatus body is minimized.

Conventional impedance matching circuits are designed to remove a reactance value of the ultrasound probe at a specific frequency. In this case, loss of the electrical energy of a reception signal in a not-considered frequency band may not be minimized. Moreover, the frequency band of the reception signal received by the ultrasound diagnosis apparatus body via a conventional impedance matching circuit may be reduced more than in the case wherein no impedance matching circuits are used.

### SUMMARY

Loss of the electrical energy of a reception signal formed by an ultrasound probe is minimized and the frequency band of the reception signal is reduced so that a reception signal corresponding to a result of the minimizations is transmitted to a body of an ultrasound diagnosis apparatus.

Loss of the electrical energy of a reception signal is minimized according to the type, application, and diagnosis application of an ultrasound probe, reduction of the frequency band of the reception signal is minimized, and a reception signal corresponding to a result of the minimizations is transmitted to a body of an ultrasound diagnosis apparatus.

Loss of the electrical energy of a reception signal is minimized according to relevant frequency characteristics of a user, and reduction of the frequency band of the reception signal is minimized, and a reception signal corresponding to a result of the minimizations is transmitted to a body of an ultrasound diagnosis apparatus.

Additional aspects will be set forth in part in the description which follows and, in part, will be apparent from the description, or may be learned by practice of the presented embodiments.

According to an aspect of an embodiment, an ultrasound diagnosis apparatus includes an ultrasound probe configured to transmit an ultrasound signal to an object and receive an ultrasound echo signal from the object to form a reception signal; a body configured to receive the reception signal from the ultrasound probe and process the reception signal; an impedance matching circuit configured to match an impedance of the ultrasound probe with an impedance of the body; and a controller configured to control the impedance matching circuit to operate as at least one filter that passes or rejects a signal of a specific frequency band, based on the impedance of the ultrasound probe, the impedance of the body, and relevant frequency characteristics of the reception signal, wherein the at least one filter is controlled to match the impedance of the ultrasound probe with the impedance of the body and is controlled so that the reception signal has the relevant frequency characteristics.

According to an aspect of another embodiment, a method of controlling an ultrasound diagnosis apparatus including an ultrasound probe and a body for processing a reception signal received from the ultrasound probe includes the operations of transmitting an ultrasound signal to an object and receiving an ultrasound echo signal from the object to form the reception signal; controlling an impedance matching circuit for matching an impedance of the ultrasound probe with an impedance of the body to operate as at least one filter that passes or rejects a signal of a specific frequency band, based on the impedance of the ultrasound probe, the impedance of the body, and relevant frequency characteristics of the reception signal; and processing a reception signal that has passed through the impedance matching circuit, wherein the reception signal that has passed through the impedance matching circuit has the relevant frequency characteristics.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram of a structure of an ultrasound diagnosis apparatus according to an embodiment;

FIG. 2 is a diagram of an ultrasound diagnosis apparatus according to an embodiment;

FIG. 3 is a block diagram of a structure of an ultrasound diagnosis apparatus according to an embodiment;

FIG. 4 is a block diagram illustrating, in detail, an impedance matching circuit included in an ultrasound diagnosis apparatus according to an embodiment;

FIG. 5 is a block diagram of a structure of an ultrasound diagnosis apparatus including an impedance matching circuit operating as a 2-stage filter, according to an embodiment;

FIG. 6 is a circuit diagram for explaining a process of determining elements that are included in an impedance matching circuit, and element values of the elements, according to an embodiment;

FIG. 7 is a circuit diagram for explaining a process of determining elements that are included in an impedance matching circuit, and element values of the elements, according to an embodiment;

FIG. 8 is a circuit diagram for explaining a process of determining elements that are included in an impedance matching circuit, and element values of the elements, according to an embodiment;

FIG. 9 is a circuit diagram a structure of an impedance matching circuit operating as a low pass filter, according to an embodiment;

FIG. 10 is a graph showing the characteristics of an impedance matching circuit according to an embodiment respectively corresponding to inductance values when the impedance matching circuit operates as a low-pass filter;

FIG. 11 is a graph showing the characteristics of an impedance matching circuit according to an embodiment respectively corresponding to capacitance values when the impedance matching circuit operates as a low-pass filter;

FIG. 12 is a graph showing the characteristics of an impedance matching circuit according to an embodiment respectively corresponding to cutoff frequencies when the impedance matching circuit operates as a low-pass filter;

FIG. 13 is a graph showing a result of impedance matching performed by using an impedance matching circuit according to an embodiment operating as a low pass filter;

FIG. 14 is a circuit diagram a structure of an impedance matching circuit operating as a high pass filter, according to an embodiment;

FIG. 15 is a graph showing the characteristics of an impedance matching circuit according to an embodiment respectively corresponding to capacitance values when the impedance matching circuit operates as a high-pass filter;

FIG. 16 is a graph showing the characteristics of an impedance matching circuit according to an embodiment respectively corresponding to inductance values when the impedance matching circuit operates as a high-pass filter;

FIG. 17 is a graph showing the characteristics of an impedance matching circuit according to an embodiment respectively corresponding to cutoff frequencies when the impedance matching circuit operates as a high-pass filter;

FIG. 18 is a graph showing a result of impedance matching performed by using an impedance matching circuit according to an embodiment operating as a high pass filter;

FIG. 19 is a block diagram of a structure of an ultrasound diagnosis apparatus including an impedance matching circuit including a plurality of circuit elements of an identical type having different values, according to an embodiment;

FIG. 20 is a circuit diagram of a structure of an ultrasound diagnosis apparatus including an impedance matching circuit including a plurality of circuit element banks, according to an embodiment;

FIG. 21 is a block diagram of a structure of an ultrasound diagnosis apparatus including an impedance matching circuit to which a plurality of ultrasound probes is connected, according to an embodiment;

FIG. 22 is a flowchart of a method of controlling an ultrasound diagnosis apparatus, according to an embodiment; and

FIG. 23 is a flowchart of a method of controlling an ultrasound diagnosis apparatus, according to an embodiment.

### DETAILED DESCRIPTION

The principle of the present invention is explained and embodiments are disclosed so that the scope of the present invention is clarified and one of ordinary skill in the art to which the present invention pertains implements the present invention. The disclosed embodiments may have various forms.

Like reference numerals refer to like elements throughout the specification. In the specification, all elements of embodiments are not explained, but general matters in the technical field of the present invention or redundant matters between embodiments will not be described. Terms ‘part’ and ‘portion’ used herein may be implemented using software or hardware, and, according to embodiments, a plurality of ‘parts’ or ‘portions’ may be implemented using a single unit or element, or a single ‘part’ or ‘portion’ may be implemented using a plurality of units or elements. The operational principle of the present invention and embodiments thereof will now be described more fully with reference to the accompanying drawings.

An image used herein may be a medical image captured by a medical imaging apparatus, such as a magnetic resonance imaging (MRI) apparatus, a computed tomography (CT) apparatus, an ultrasound imaging apparatus, or an X-ray apparatus.

Throughout the specification, a term ‘object’ is a thing to be imaged, and may include a human, an animal, or a part of a human or animal. For example, the object may include a part of a body (i.e., an organ), a phantom, or the like.

Throughout the specification, an “ultrasound image” refers to an image of an object processed based on ultrasound signals transmitted to the object and reflected therefrom.

Throughout the specification, a “relevant frequency” refers to a frequency necessary for image generation when an ultrasound diagnosis apparatus body generates an image by processing a reception signal received via an ultrasound probe.

Embodiments now will be described more fully hereinafter with reference to the accompanying drawings.

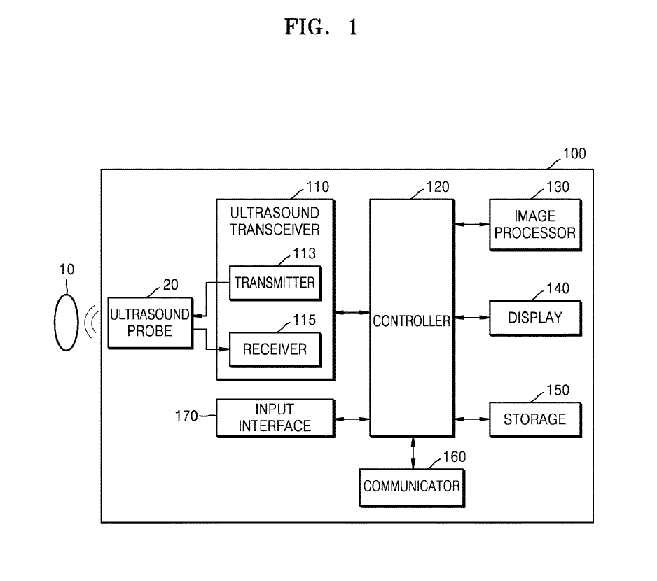

FIG. 1 is a block diagram showing a configuration of an ultrasound diagnosis apparatus 100 according to an embodiment. An ultrasound diagnosis apparatus 100 according to an embodiment may include an ultrasound probe 20, an ultrasound transceiver 110, a controller 120, an image processor 130, a display 140, a storage 150, a communicator 160, and an input interface 170.

The ultrasound diagnosis apparatus 100 may be of a cart-type or a portable-type ultrasound diagnosis apparatus. Examples of the portable-type ultrasound imaging apparatus 200 may include a smartphone, a laptop computer, a personal digital assistant (PDA), and a tablet personal computer (PC), each of which may include a probe and an application, but embodiments are not limited thereto.

The ultrasound probe 20 may include a plurality of transducers. The plurality of transducers may transmit ultrasound signals to an object 10 in response to transmitting signals applied by a transmitter 113. The plurality of transducers may receive ultrasound signals reflected from the object 10 to generate reception signals. In addition, the ultrasound probe 20 and the ultrasound diagnosis apparatus 100 may be formed in one body, or the ultrasound probe 20 and the ultrasound diagnosis apparatus 100 may be formed separately but linked wirelessly or via wires. In addition, the ultrasound diagnosis apparatus 100 may include one or more probes 20 according to embodiments.

The controller 120 may control the transmitter 113 to generate transmitting signals to be applied to each of the plurality of transducers based on a position and a focal point of the plurality of transducers included in the ultrasound probe 20.

The controller 120 may control the ultrasound receiver 115 to generate ultrasound data by converting reception signals received from the ultrasound probe 20 from analogue to digital signals and summing the digital reception signals based on a position and a focal point of the plurality of transducers.

The image processor 130 may generate an ultrasound image by using ultrasound data generated from the ultrasound receiver 115.

The display 140 may display the generated ultrasound image and various pieces of information processed by the ultrasound diagnosis apparatus 100. The ultrasound diagnosis apparatus 100 may include two or more displays 140 according to embodiments. The display 140 may include a touch screen in combination with a touch panel.

The controller 120 may control the operations of the ultrasound diagnosis apparatus 100 and flow of signals between the internal elements of the ultrasound diagnosis apparatus 100. The controller 120 may include a memory for storing a program or data to perform functions of the ultrasound diagnosis apparatus 100 and a processor and/or a microprocessor (not shown) for processing the program or data. For example, the controller 120 may control the operation of the ultrasound diagnosis apparatus 100 by receiving a control signal from the input interface 170 or an external apparatus.

The ultrasound diagnosis apparatus 100 may include the communicator 160 and may be connected to external apparatuses, for example, servers, medical apparatuses, and portable devices such as smart phones, tablet personal computers (PCs), wearable devices, etc., via the communicator 160.

The communicator 160 may include at least one element capable of communicating with the external apparatus. For example, the communicator 160 may include at least one among a short-range communication module, a wired communication module, and a wireless communication module.

The communicator 160 may receive a control signal and data from an external apparatus and transmit the received control signal to the controller 120, so that the controller 120 may control the ultrasound diagnosis apparatus 100 in response to the received control signal.

The controller 120 may transmit a control signal to an external apparatus via the communicator 160 so that the external apparatus may be controlled in response to the control signal of the controller 220.

For example, the external apparatus connected to the ultrasound diagnosis apparatus 100 may process data of the external apparatus in response to control signal of the controller 220 received via the communicator 160.

A program for controlling the ultrasound diagnosis apparatus 100 may be installed in the external apparatus. The program may include command languages to perform part of operation of the controller 120 or the entire operation of the controller 220.

The program may be pre-installed in the external apparatus or may be installed by a user of the external apparatus by downloading the program from a server that provides applications. The server that provides applications may include a recording medium where the program is stored.

The storage 150 may store various data or programs for driving and controlling the ultrasound diagnosis apparatus 100, input and/or output ultrasound data, ultrasound images, applications, etc.

The input interface 170 may receive a user's input to control the ultrasound diagnosis apparatus 100 and may include a key pad, button, keypad, mouse, trackball, jog switch, knob, a touchpad, a touch screen, a microphone, a motion input means, a biometrics input means, etc. For example, the user's input may include inputs for manipulating buttons, keypads, mice, track balls, jog switches, or knobs, inputs for touching a touchpad or a touch screen, a voice input, a motion input, and a bioinformation input, for example, iris recognition or fingerprint recognition, but an exemplary embodiment is not limited thereto.

The storage 150 may store information about the ultrasound probe 20 and a body of the ultrasound diagnosis apparatus 100. According to an embodiment, the storage 150 may store information about the type of ultrasound probe 20, identification information for identifying the type of ultrasound probe 20, and information about applications of the ultrasound probe 20.

The storage 150 may also store information about the impedance of the ultrasound probe 20. According to an embodiment, the storage 150 may store information about impedance corresponding to the type of ultrasound probe 20. In detail, the ultrasound probe 20 may include an ultrasound probe including a single transducer element, and an ultrasound probe including a transducer array comprised of a plurality of transducer elements.

The storage 150 may store information about the impedance of an ultrasound probe including a single transducer element. In this case, a plurality of ultrasound probes each including a single transducer element may be different in terms of impedance, and the storage 150 may store information about the respective impedances of the plurality of ultrasound probes different from one another in terms of impedance. A plurality of ultrasound probes each including a transducer array may be different in terms of impedance, and the storage 150 may store information about the respective impedances of the plurality of ultrasound probes that are different from one another in terms of impedance.

The storage 150 may store information about an impedance matching circuit corresponding to the information about the impedance corresponding to the type of ultrasound probe 20 and information about the impedance of the body. According to an embodiment, the storage 150 may store information about elements that are included in an impedance matching circuit that matches the impedance of the body with the impedance of the ultrasound probe 20. In this case, the storage 150 may store information about the types of elements that are included in the impedance matching circuit, and information about element values of the elements that are included in the impedance matching circuit.

The storage 150 may store information about elements that are included in an impedance matching circuit corresponding to the relevant frequency characteristics of a reception signal applied to the body. In this case, the information about the elements that are included in the impedance matching circuit may include information about the types of the elements, information about the element values of the elements, and information about a method of connecting the elements. According to an embodiment, the storage 150 may include information about elements that are included in an impedance matching circuit corresponding to a cutoff frequency of an impedance matching circuit that operates as a filter. In this case, the storage 150 may include information about elements that are included in an impedance matching circuit respectively corresponding to a plurality of cutoff frequencies.

The storage 150 may store diagnosis applications executable by the ultrasound diagnosis apparatus 100. According to an embodiment, the storage 150 may store diagnosis applications corresponding to parts of a human body. According to an embodiment, the diagnosis applications stored in the storage 150 may be applications regarding abdominal, musculoskeletal, heart, and ob/gyn diagnoses. In this case, the abdominal diagnosis application stored in the storage 150 may include diagnosis applications regarding thyroid, liver, kidney, heart, stomach, pancreas, gallbladder, spleen, esophagus, large intestine, small intestine, and rectum, and the musculoskeletal diagnosis application stored in the storage 150 may include diagnosis applications regarding respective muscles and blood vessels (such as carotid and aorta) of parts of a human body.

The storage 150 may store at least one image processing application capable of processing and displaying various types of images, such as a planar image and a three-dimensional (3D) image.

The storage 150 may store relevant frequency characteristics of reception signals corresponding to applications stored therein. The storage 150 may also include information about elements that are included in an impedance matching circuit corresponding to the relevant frequency characteristics.

The communicator 160 may transmit or receive the information about the ultrasound probe 20 and the ultrasound diagnosis apparatus body to or from an external apparatus. According to an embodiment, the communicator 160 may transmit or receive the information about the type of ultrasound probe 20, the identification information for identifying the type of ultrasound probe 20, and the information about applications of the ultrasound probe 20 to or from an external apparatus.

According to an embodiment, the communicator 160 may transmit or receive the information about the impedance of the ultrasound probe 20 to or from an external apparatus.

According to an embodiment, the communicator 160 may transmit or receive the information about the impedance matching circuit corresponding to the information about the impedance corresponding to the type of ultrasound probe 20 and the information about the impedance of the body to or from an external apparatus.

According to an embodiment, the communicator 160 may transmit or receive the information about the elements that are included in the impedance matching circuit that matches the impedance of the body with the impedance of the ultrasound probe 20, to or from an external apparatus. In this case, the communicator 160 may transmit or receive the information about the types of elements that are included in the impedance matching circuit, and the information about the element values of the elements that are included in the impedance matching circuit, to or from an external apparatus.

The communicator 160 may transmit or receive the information about the elements that are included in the impedance matching circuit corresponding to the relevant frequency characteristics of the reception signal applied to the body to or from an external apparatus.

The communicator 160 may transmit or receive the information about the applications executed by the ultrasound diagnosis apparatus 100 to or from an external apparatus. The communicator 160 may transmit or receive the information about the relevant frequency characteristics of the reception signals corresponding to the applications stored in the ultrasound diagnosis apparatus 100, to or from an external apparatus.

The input interface 170 may receive a user input for selecting a type of the ultrasound probe 20. The input interface 170 may receive a user input of selecting one from a plurality of applications. The input interface 170 may receive a user input of selecting one from a plurality of ultrasound probes connected to the body. The input interface 170 may receive a user input of changing the relevant frequency characteristics of a reception signal input to the body. The input unit 170 may receive a user input of changing the cutoff frequency of an impedance matching circuit.

The controller 120 may control measurement of the impedance of the ultrasound probe 20 connected to the body of the ultrasound diagnosis apparatus 100. The controller 120 may control measurement of the impedance of the body of the ultrasound diagnosis apparatus 100. The controller 120 may control an impedance matching circuit to match the measured impedance of the ultrasound probe 20 with the measured impedance of the body of the ultrasound diagnosis apparatus 100.

The controller 120 may control the impedance matching circuit to match the impedance of the ultrasound probe 20 with the impedance of the body of the ultrasound diagnosis apparatus 100. According to an embodiment, the controller 120 may control the impedance matching circuit to act as at least one filter that passes or stops a specific frequency band of a reception signal that passes through the impedance matching circuit. In this case, the controller 120 may control the impedance matching circuit so that the reception signal that passes through the impedance matching circuit includes relevant frequency characteristics. The controller 120 may control the impedance matching circuit to operate as a multi-stage filter.

The controller 120 may select circuit elements that are included in the impedance matching circuit. According to an embodiment, the controller 120 may select the types of circuit elements that are included in the impedance matching circuit, a method of connecting the circuit elements, and the values of the circuit elements.

The controller 120 may select at least one circuit element from among a plurality of circuit elements of an identical type, and control the impedance matching circuit to match the impedance of the ultrasound probe 20 with the impedance of the body by using the selected circuit element.

The controller 120 may select at least one circuit element from among a plurality of circuit elements of different types, and control the impedance matching circuit to match the impedance of the ultrasound probe 20 with the impedance of the body by using the selected circuit element.

The controller 120 may select at least one circuit element from a plurality of circuit element banks of different types each including circuit elements of an identical type having different values, and control the impedance matching circuit to match the impedance of the ultrasound probe 20 with the impedance of the body by using the selected circuit element.

The controller 120 may select circuit elements constituting an impedance matching circuit, based on a user input of changing relevant frequency characteristics, and control the impedance matching circuit to match the impedance of the ultrasound probe 20 with the impedance of the body by using the selected circuit elements.

The controller 120 may detect an ultrasound probe currently-being-used by a user from among a plurality of ultrasound probes connected to the body. According to an embodiment, the controller 120 may transmit an ultrasound signal to an object and detect an ultrasound probe that is currently receiving an ultrasound echo signal from the object, thereby detecting the ultrasound probe currently-being-used by the user. According to another embodiment, the controller 120 may detect the ultrasound probe currently-being-used by the user, by detecting movements of the ultrasound probes. According to another embodiment, the controller 120 may detect the ultrasound probe currently-being-used by the user, by detecting whether the ultrasound probes have been separated from a probe holder. In this case, a radio frequency identification (RFID) reader may be included in at least one of an ultrasound probe and the probe holder, and an RFID writer may be included in the other one. The controller 120 may detect whether the ultrasound probes have been separated from a probe holder, by using the RFID reader and the RFID writer.

The controller 120 may control the impedance matching circuit to change the relevant frequency characteristics of the reception signal that passes through the impedance matching circuit, according to an application currently being executed by the ultrasound diagnosis apparatus 100. The controller 120 may control the impedance matching circuit to change the relevant frequency characteristics of the reception signal passing through the impedance matching circuit, in response to a user input of changing the application currently being executed by the ultrasound diagnosis apparatus 100.

According to an embodiment, the controller 120 may control the impedance matching circuit to operate as a high-pass filter, in response to a user input of selecting applications used for musculoskeletal, blood vessel, and thyroid diagnoses. The relevant frequency characteristics of the reception signal corresponding to the applications used for musculoskeletal and blood vessel diagnoses include a high frequency. In detail, as the frequency of an ultrasound signal transmitted by a human body increases, the resolution thereof increases. However, the ultrasound signal does not pass deeply through the human body. Thus, ultrasound waves used for the skeleton and musculature and blood vessels located on the surface of a human body use a high-frequency ultrasound signal. In general, ultrasound signals for use in musculoskeletal and blood vessel diagnoses include a frequency of 4 to 11MHz and are transmitted by a human body via a linear-type ultrasound probe.

According to an embodiment, the controller 120 may control the impedance matching circuit to operate as a low-pass filter, in response to a user input of selecting applications for use in abdominal, heart, and ob/gyn diagnoses. The relevant frequency characteristics of a reception signal corresponding to the applications used for abdominal, heart, and ob/gyn diagnoses include a low frequency. In detail, as the frequency of an ultrasound signal transmitted by a human body decreases, the resolution thereof decreases. However, the ultrasound signal may pass deeply through the human body. Thus, ultrasound waves used for diagnosing abdomen and heart located deeply in a human body or for ob/gyn diagnosis use a low-frequency ultrasound signal. In general, ultrasound signals for abdomen, heart, and ob-gyn include a frequency of 3 to 5 MHz and are transmitted by a human body via a curved-type ultrasound probe.

The controller 120 may detect the type and application of the ultrasound probe 20 connected to the ultrasound diagnosis apparatus 100. According to an embodiment, the controller 120 may detect the type of ultrasound probe 20, based on the identification information of the ultrasound probe 20 stored in the storage 150. The controller 120 may select circuit elements that are included in an impedance matching circuit corresponding to the type of ultrasound probe 20, and control the impedance matching circuit to match the impedance of the ultrasound probe 20 with the impedance of the body by using the selected circuit elements.

According to an embodiment, the type of ultrasound probe 20 may vary according to use purposes. In this case, ultrasound probes may be classified into linear-type ultrasound probes, curved-type ultrasound probes, and sector-type ultrasound probes.

The linear-type ultrasound probes may be used to diagnose tissues located near the skin, such as the skeleton and musculature, blood vessels, and thyroid. Because tissues located near the skin are diagnosed using a high-frequency ultrasound signal as described above, when the controller 120 detects a linear-type ultrasound probe, the controller 120 may control the impedance matching circuit to operate as a high-pass filter.

The curved-type ultrasound probes may be used to diagnose tissues located deeply within the human body, such as abdomen, heart, and fetus. In detail, the curved-type ultrasound probes may be used to diagnose liver, kidney, heart, stomach, pancreas, gallbladder, spleen, esophagus, large intestine, small intestine, and rectum. Because tissues located deeply within the human body are diagnosed using a low-frequency ultrasound signal as described above, when the controller 120 detects a curved-type ultrasound probe, the controller 120 may control the impedance matching circuit to operate as a low-pass filter.

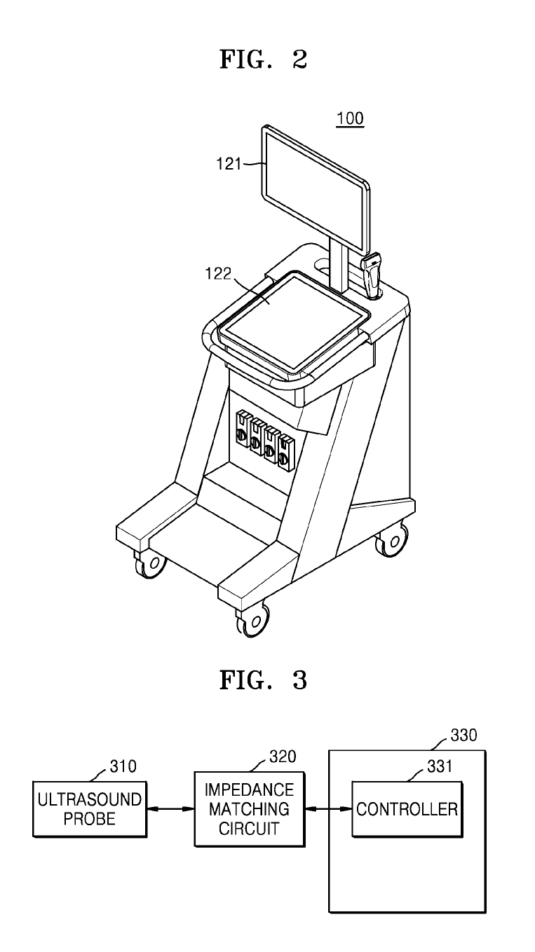

An example of the ultrasound diagnosis apparatus 100 according to an embodiment will now be described with reference to FIG. 2.

FIG. 2 is a diagram of the ultrasound diagnosis apparatus 100 according to an embodiment.

Referring to FIG. 2, the ultrasound diagnosis apparatus 100 may include a main display 121 and a sub-display 122. One of the main display 121 and the sub-display 122 may be implemented using a touch screen. The main display 121 and the sub-display 122 may display an ultrasound image or various pieces of information that are processed by the ultrasound diagnosis apparatus 100. The main display 121 and the sub-display 122 may be implemented using touch screens, and may provide graphical user interfaces (GUIs) to receive data for controlling the ultrasound diagnosis apparatus 100 from a user. For example, the main display 121 may display an ultrasound image, and the sub-display 122 may display a control panel for controlling display of the ultrasound image, in a GUI form. The sub-display 122 may receive data for controlling display of an image, via a control panel displayed in a GUI form. The ultrasound diagnosis apparatus 100 may control display of an ultrasound image displayed on the main display 121, by using the received control data.

Referring to FIG. 2, the ultrasound diagnosis apparatus 100 may include a plurality of ultrasound probes connected to a body of the ultrasound diagnosis apparatus 100. In detail, the plurality of ultrasound probes may be of different types. The plurality of ultrasound probes may be used for different purposes. The plurality of ultrasound probes may have different impedance magnitudes. The plurality of ultrasound probes may generate reception signals having different prevalent frequency characteristics.

The plurality of ultrasound probes may be connected to the body via impedance matching circuits, respectively. In this case, separate impedance matching circuits may be used in correspondence with the plurality of ultrasound probes, respectively. The impedance matching circuits may be connected to the plurality of ultrasound probes via a plurality of input ports and may be connected to the body via a single output port.

FIG. 3 is a block diagram of a structure of an ultrasound diagnosis apparatus according to an embodiment.

According to the embodiment of FIG. 3, the ultrasound diagnosis apparatus may include an ultrasound probe 310, an impedance matching circuit 320, and a body 330.

The ultrasound probe 310 may transmit an ultrasound signal to an object and may receive an ultrasound echo signal from the object. The ultrasound probe 310 may form a reception signal by using the received ultrasound echo signal. The reception signal formed by the ultrasound probe 310 may be input to the body 330 via the impedance matching circuit 320.

The impedance matching circuit 320 may match impedance of the ultrasound probe 310 with impedance of the body 330 to minimize electrical energy loss of the reception signal input to the body 330. The impedance matching circuit 320 may operate as a filter that transmits or blocks a specific frequency band of the reception signal that passes through the impedance matching circuit 320. The impedance matching circuit 320 may be included in the body 330 or in the ultrasound probe 310. A part of the impedance matching circuit 320 may be included in the ultrasound probe 310, and the remaining part thereof may be included in the body 330.

The body 330 may process the received reception signal. The body 330 may process the received reception signal to generate an ultrasound diagnosis image. The body 330 may include a controller 331 that controls overall operations of the ultrasound diagnosis apparatus. The controller 331 may select circuit elements that are included in the impedance matching circuit 320. The controller 331 may select circuit elements that are included in the impedance matching circuit 320, based on the impedance of the ultrasound probe 310, the impedance of the body 330, and relevant frequency characteristics of the reception signal. The controller 331 may control the reception signal passing through the impedance matching circuit 320 to include relevant frequency characteristics by using the selected circuit elements. According to an embodiment, the controller 331 may select circuit elements that are included in the impedance matching circuit 320, so that the impedance matching circuit 320 operates as one of a high-pass filter, a low-pass filter, a band-pass filter, and a band-stop filter in accordance with the relevant frequency characteristics.

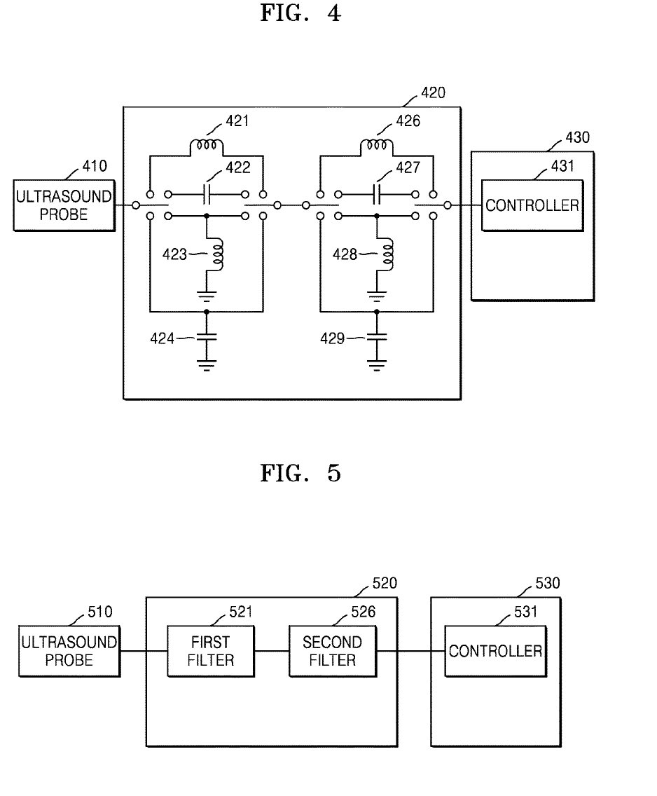

FIG. 4 is a block diagram illustrating, in detail, an impedance matching circuit 420 included in an ultrasound diagnosis apparatus according to an embodiment.

According to the embodiment of FIG. 4, the impedance matching circuit 420 may include a plurality of circuit elements of different types. The impedance matching circuit 420 may include a switch or multiplexer (MUX) that connects the circuit elements that are included in the impedance matching circuit 420.

The circuit elements included in the impedance matching circuit 420 may be variable elements of which element values are variable. According to an embodiment, a mechanical tab may be installed on a main coil, and inductance of a variable inductor may vary according to locations of the mechanical tab. The variable inductor may change its inductance by using a ratio between the main coil and an auxiliary coil installed together with the variable inductor. The inductance of the variable inductor may be changed according to the amount of current applied to the variable inductor by using a magnetic core formed of different types of magnetic materials. However, the variable inductor is not limited thereto. According to an embodiment, a variable capacitor has a capacitance that may be changed according to a voltage applied to the variable capacitor, but embodiments are not limited thereto.

The circuit elements that are included in the impedance matching circuit 420 may include a serial inductor, a serial capacitor, a parallel inductor, and a parallel capacitor. The circuit elements that are included in the impedance matching circuit 420 may be selected by using a switch.

A controller 431 may select at least one from circuit elements 421, 422, 423, 424, 426, 427, 428, and 429 that are included in the impedance matching circuit 420, and may control the impedance matching circuit 420 to be reconstructed with the selected circuit element.

The controller 431 may select an array of the circuit elements that are included in the impedance matching circuit 420, according to an impedance magnitude of an ultrasound probe 410 and an impedance magnitude of a body 430. According to an embodiment, when the ultrasound probe 410 includes a single transducer element, because the impedance of the ultrasound probe 410 is smaller than the impedance of the body 430, the controller 431 may match the impedance of the ultrasound probe 410 with the impedance of the body 430 by selecting the serial inductor 421 and the parallel capacitor 429 from the circuit elements that are included in the impedance matching circuit 420. The controller 431 may match the impedance of the ultrasound probe 410 with the impedance of the body 430 by selecting the serial capacitor 422 and the parallel inductor 428 from the circuit elements that are included in the impedance matching circuit 420. According to an embodiment, when the ultrasound probe 410 includes a transducer array of a plurality of transducer elements, because the impedance of the ultrasound probe 410 is larger than the impedance of the body 430, the controller 431 may match the impedance of the ultrasound probe 410 with the impedance of the body 430 by selecting the parallel inductor 423 and the serial capacitor 427 from the circuit elements that are included in the impedance matching circuit 420. Alternatively, the controller 431 may match the impedance of the ultrasound probe 410 with the impedance of the body 430 by selecting the parallel capacitor 424 and the serial inductor 426 from the circuit elements that are included in the impedance matching circuit 420.

The controller 431 may select circuit elements that are included in the impedance matching circuit 420, according to relevant frequency characteristics of a reception signal that passes through the impedance matching circuit 420. According to an embodiment, the controller 431 may select circuit elements of the impedance matching circuit 420 so that the impedance matching circuit 420 operates as a low-pass filter for spurious removal or harmonic removal of the reception signal. The controller 431 may control the impedance matching circuit 420 to operate as a low-pass filter, by selecting the serial inductor 421 and the parallel capacitor 429 from the circuit elements that are included in the impedance matching circuit 420. According to an embodiment, the controller 431 may select circuit elements of the impedance matching circuit 420 so that the impedance matching circuit 420 operates as a high-pass filter for low-frequency oscillation prevention and ringing prevention of the reception signal. The controller 431 may control the impedance matching circuit 420 to operate as a high-pass filter, by selecting the serial capacitor 422 and the parallel inductor 428 from the circuit elements that are included in the impedance matching circuit 420.

According to an embodiment, when the impedance matching circuit 420 operates as a low-pass filter, the controller 431 may select the serial inductor 421 and the parallel capacitor 429 from the circuit elements that are included in the impedance matching circuit 420. The controller 431 may control a switch to connect the selected serial inductor 421 to the ultrasound probe 410. The controller 431 may control a switch to connect the selected serial inductor 421 to the selected parallel capacitor 429. The controller 431 may control a switch to connect the selected parallel capacitor 429 to the body 430.

According to an embodiment, when the impedance matching circuit 420 operates as a high-pass filter, the controller 431 may select the serial capacitor 422 and the parallel inductor 428 from the circuit elements that are included in the impedance matching circuit 420. The controller 431 may control a switch to connect the selected serial capacitor 422 to the ultrasound probe 410. The controller 431 may control a switch to connect the selected serial capacitor 422 to the selected parallel inductor 428. The controller 431 may control a switch to connect the selected parallel inductor 428 to the body 430.

According to an embodiment, the controller 431 may select circuit elements that are included in the impedance matching circuit 420, according to the impedance of the ultrasound probe 410 and the impedance of the body 430. In detail, when the ultrasound probe 410 includes a single transducer element and thus the impedance of the ultrasound probe 410 is smaller than the impedance of the body 430, the controller 431 may select the serial circuit elements 421 and 422 and the parallel circuit elements 428 and 429. The controller 431 may control a switch to connect the selected serial circuit elements 421 and 422 to the ultrasound probe 410. The controller 431 may control a switch to connect the selected serial circuit elements 421 and 422 to the selected parallel circuit elements 428 and 429. The controller 431 may control a switch to connect the selected parallel circuit elements 428 and 429 to the body 430.

On the other hand, when the ultrasound probe 410 includes a transducer array of a plurality of transducer elements and thus the impedance of the ultrasound probe 410 is larger than the impedance of the body 430, the controller 431 may select the parallel circuit elements 423 and 424 and the serial circuit elements 426 and 427. The controller 431 may control a switch to connect the selected parallel circuit elements 423 and 424 to the ultrasound probe 410. The controller 431 may control a switch to connect the selected parallel circuit elements 423 and 424 to the selected serial circuit elements 426 and 427. The controller 431 may control a switch to connect the selected serial circuit elements 426 and 427 to the body 430.

The controller 431 may control the types and element values of the circuit elements that are included in the impedance matching circuit 420 operating as a filter, thereby controlling a cutoff frequency of the filter.

A method in which the controller 431 selects the types and element values of the circuit elements that are included in the impedance matching circuit 420 will be described later.

FIG. 5 is a block diagram of a structure of an ultrasound diagnosis apparatus including an impedance matching circuit 520 operating as a 2-stage filter, according to an embodiment.

According to the embodiment of FIG. 5, the impedance matching circuit 520 may operate as a multi-stage filter. According to an embodiment, the impedance matching circuit 520 may operate as a 2-stage filter or as a filter with three or more stages.

According to an embodiment, a first filter 521 may be a low-pass filter and a second filter 526 may be a high-pass filter. In this case, the impedance matching circuit 520 may operate as a band-pass filter or a band-stop filter. According to another embodiment, the first filter 521 may be a high-pass filter and the second filter 526 may be a low-pass filter. In this case, the impedance matching circuit 520 may operate as a band-pass filter or a band-stop filter. According to another embodiment, both the first filter 521 and the second filter 526 may be low-pass filters. In this case, a high frequency band of a reception signal that passes through the impedance matching circuit 520 may be securely removed via the impedance matching circuit 520. According to another embodiment, both the first filter 521 and the second filter 526 may be high-pass filters. In this case, a low frequency band of a reception signal that passes through the impedance matching circuit 520 may be securely removed via the impedance matching circuit 520.

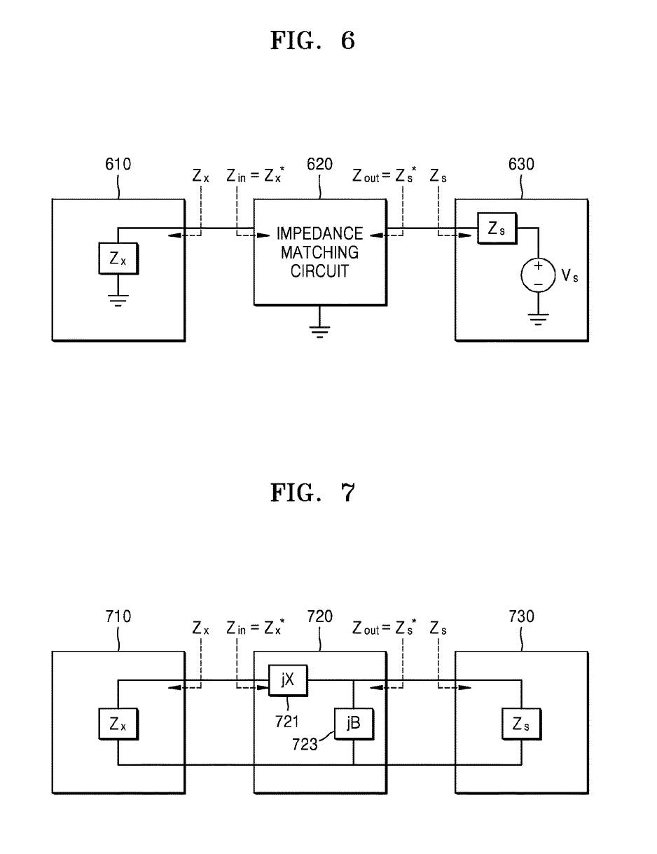

FIG. 6 is a circuit view for explaining a process of determining elements that are included in an impedance matching circuit 620, and element values of the elements, according to an embodiment.

According to the embodiment of FIG. 6, the impedance matching circuit 620 may be located between an ultrasound probe 610 and a body 630. In order for the impedance matching circuit 620 to match impedance of the ultrasound probe 610 with impedance of the body 630, an input impedance Zin of the impedance matching circuit 620 needs to be equal to a conjugated complex number ZX* of impedance ZX of the ultrasound probe 610, and an output impedance Zout of the impedance matching circuit 620 needs to be equal to a conjugated complex number Zs* of impedance Zs of the body 630.

The impedance ZX of the ultrasound probe 610 is calculated using Equation

Z_{X}R_{X}(ω)+fX_{X}(ω)=a+jb, [Equation 1]

The impedance Zs of the body 630 is calculated using Equation 2.

Z_{S}−R_{S}(ω)+jX_{S}(ω)−c+jd [Equation 2]

The structure of the impedance matching circuit 620 may be implemented in two types according to the magnitudes of the impedance of the ultrasound probe 610 and the impedance of the body 630.

FIG. 7 is a circuit view for explaining a process of determining elements that are included in an impedance matching circuit 720, and element values of the elements, according to an embodiment.

According to the embodiment of FIG. 7, an impedance magnitude of an ultrasound probe 710 is smaller than an impedance magnitude of a body 730 (i.e., |Z_{S}|>|Z_{X}|), the impedance matching circuit 720 is implemented such that a serial circuit element is connected to the ultrasound probe 710 and a parallel circuit element is connected to the body 730.

The types and element values of devices that are included in the impedance matching circuit 720 are calculated using Equation 3 and Equation 4.

$$\begin{matrix}{Z}_{in} = {Z}_{X}^{*} = a - jb & [ Equation 3 ] \\ {Z}_{in} = jX + ( jB ‖ {Z}_{s} ) = jX + \frac{jB \cdot ( c + jd )}{jB + ( c + jd )} = \frac{c \cdot {B}^{2}}{{c}^{2} + {( d + B )}^{2}} - j \frac{d \cdot {B}^{2} + ( {c}^{2} + {d}^{2} ) \cdot B + \{ {c}^{2} + {( d + B )}^{2} \} \cdot X}{{c}^{2} + {( d + B )}^{2}} & [ Equation 4 ]\end{matrix}$$

When the right side of Equation 3 is the same as that of Equation 4, impedance of the ultrasound probe 710 is matched with impedance of the body 730.

Therefore, when the right sides of Equations 3 and 4 are arranged to be equal to each other, Equation 5 and Equation 6 are obtained as follows.

$$\begin{matrix}a = \frac{c \cdot {B}^{2}}{{c}^{2} + {( d + B )}^{2}} & [ Equation 5 ] \\ b = - \frac{d \cdot {B}^{2} + ( {c}^{2} + {d}^{2} ) \cdot B + \{ {c}^{2} + {( d + B )}^{2} \} \cdot X}{{c}^{2} + {( d + B )}^{2}} & [ Equation 6 ]\end{matrix}$$

The types and element values of elements that are included in the impedance matching circuit 720 are calculated using Equation 7 and Equation 8.

$$\begin{matrix}{Z}_{out} = {Z}_{S}^{*} = c - jd & [ Equation 7 ] \\ {Z}_{out} = jB ‖ ( jX + {Z}_{X} ) = jB ‖ ( jX + c + jd ) = \frac{a \cdot {B}^{2}}{{a}^{2} + {( b + B + X )}^{2}} - j \frac{{a}^{2} \cdot B + ( b + X ) \cdot ( b + B + X ) \cdot B}{{a}^{2} + {( b + B + X )}^{2}} & [ Equation 8 ]\end{matrix}$$

When the right side of Equation 7 is the same as that of Equation 8, the impedance of the ultrasound probe 710 is matched with the impedance of the body 730.

Therefore, when the right sides of Equations 7 and 8 are arranged to be equal to each other, results of Equation 9 and Equation 10 are obtained.

$$\begin{matrix}c = \frac{a \cdot {B}^{2}}{{a}^{2} + {( b + B + X )}^{2}} & [ Equation 9 ] \\ d = - \frac{{a}^{2} \cdot B + ( b + X ) \cdot ( b + B + X ) \cdot B}{{a}^{2} + {( b + B + X )}^{2}} & [ Equation 10 ]\end{matrix}$$

When element values X and B of elements that are included in the impedance matching circuit 720 are obtained using Equations 5, 6, 9 and 10, the element values X and B are equal to Equations 11 and 12.

$$\begin{matrix}B = \frac{- ad \pm \sqrt{ac ( {c}^{2} + {d}^{2} - ac )}}{a - c} & [ Equation 11 ] \\ X = \frac{- bc \pm \sqrt{ac ( {c}^{2} + {d}^{2} - ac )}}{c} & [ Equation 12 ]\end{matrix}$$

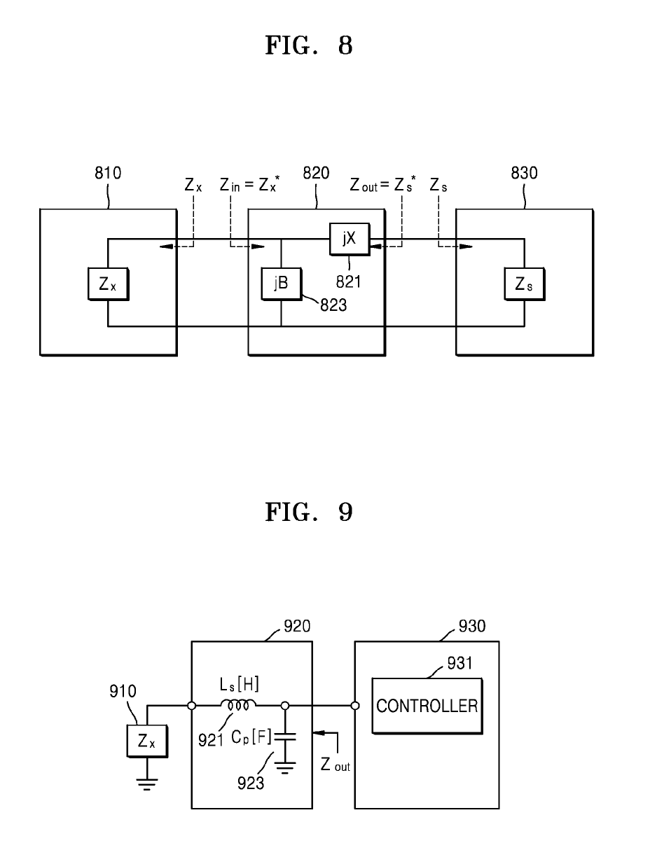

FIG. 8 is a circuit view for explaining a process of determining elements that are included in an impedance matching circuit 820, and element values of the elements, according to an embodiment.

According to the embodiment of FIG. 8, an impedance magnitude (|Zₓ|) of an ultrasound probe 810 is larger than an impedance magnitude |Z_{S}| of a body 830 (i.e., |Z_{S}|<|Zₓ|), the impedance matching circuit 820 is implemented such that a parallel circuit element is connected to the ultrasound probe 810 and a serial circuit element is connected to the body 830.

The types and element values of elements that are included in the impedance matching circuit 820 are calculated using Equation 13 and Equation 14.

$$\begin{matrix}{Z}_{in} = {Z}_{x}^{*} = a - jb & [ Equation 13 ] \\ {Z}_{in} = jB ‖ ( jX + {Z}_{s} ) = jB ‖ ( jX + c + jd ) = \frac{c \cdot {B}^{2}}{{c}^{2} + {( d + B + X )}^{2}} - j \frac{{c}^{2} \cdot B + ( d + X ) \cdot ( d + B + X ) \cdot B}{{c}^{2} + {( d + B + X )}^{2}} & [ Equation 14 ]\end{matrix}$$

When the right side of Equation 13 is the same as that of Equation 14, the impedance of the ultrasound probe 810 is matched with the impedance of the body 830.

Therefore, when the right sides of Equations 13 and 14 are arranged to be equal to each other, results of Equation 15 and Equation 16 are obtained.

$$\begin{matrix}a = \frac{c \cdot {B}^{2}}{{c}^{2} + {( d + B + X )}^{2}} & [ Equation 15 ] \\ b = - \frac{{c}^{2} \cdot B + ( d + X ) \cdot ( d + B + X ) \cdot B}{{c}^{2} + {( d + B + X )}^{2}} & [ Equation 16 ]\end{matrix}$$

The types and element values of elements that are included in the impedance matching circuit 820 are calculated using Equation 17 and Equation 18.

$$\begin{matrix}{Z}_{out} = {Z}_{S}^{*} = c - jd & [ Equation 17 ] \\ {Z}_{out} = jX + ( jB ‖ {Z}_{X} ) = jX + \frac{jB \cdot ( a + jb )}{jB + ( a + jb )} = \frac{a \cdot {B}^{2}}{{a}^{2} + {( b + B )}^{2}} - j \frac{b \cdot {B}^{2} + ( {a}^{2} + {b}^{2} ) + \{ {a}^{2} + {( b + B )}^{2} \} \cdot X}{{a}^{2} + {( b + B )}^{2}} & [ Equation 18 ]\end{matrix}$$

When the right side of Equation 17 is the same as that of Equation 18, the impedance of the ultrasound probe 810 is matched with the impedance of the body 830.

Therefore, when the right sides of Equations 17 and 18 are arranged to be equal to each other, results of Equation 19 and Equation 20 are obtained.

$$\begin{matrix}c = \frac{a \cdot {B}^{2}}{{a}^{2} + {( b + B )}^{2}} & [ Equation 19 ] \\ d = - \frac{b \cdot {B}^{2} + ( {a}^{2} + {b}^{2} ) \cdot B + \{ {a}^{2} + {( b + B )}^{2} \} \cdot X}{{a}^{2} + {( b + B )}^{2}} & [ Equation 20 ]\end{matrix}$$

When element values X and B of elements that are included in the impedance matching circuit 820 are obtained using Equations 15, 16, 19 and 20, the element values X and B are equal to Equations 21 and 22.

$$\begin{matrix}B = \frac{- bc \pm \sqrt{ac ( {a}^{2} + {b}^{2} - ac )}}{c - a} & [ Equation 21 ] \\ X = \frac{- ad \pm \sqrt{ac ( {a}^{2} + {b}^{2} - ac )}}{a} & [ Equation 22 ]\end{matrix}$$

When the element values X and B of the elements that are included in the impedance matching circuit 820, which are calculated using Equations 21 and 22, are positive values, the elements are inductors. On the other hand, when the element values X and B of the elements that are included in the impedance matching circuit 820, which are calculated using Equations 21 and 22, are negative values, the elements are capacitors.

In order for the impedance matching circuits 720 and 820 to operate as filters that pass or stop specific frequency bands of reception signals that pass through the impedance matching circuits 720 and 820, the element values of Equations 11 and 12 need to have opposite signs, and the element values of Equations 21 and 22 need to have opposite signs. In other words, circuit elements having element values and constructing the impedance matching circuits 720 and 820 are different types of elements. When different types of circuit elements are included in the impedance matching circuits 720 and 820, the impedance matching circuits 720 and 820 may operate as filters that pass or stop specific frequency bands.

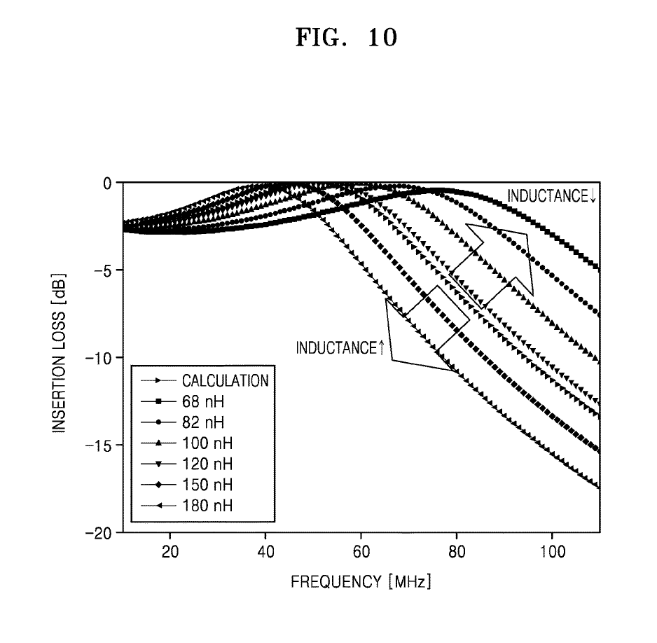

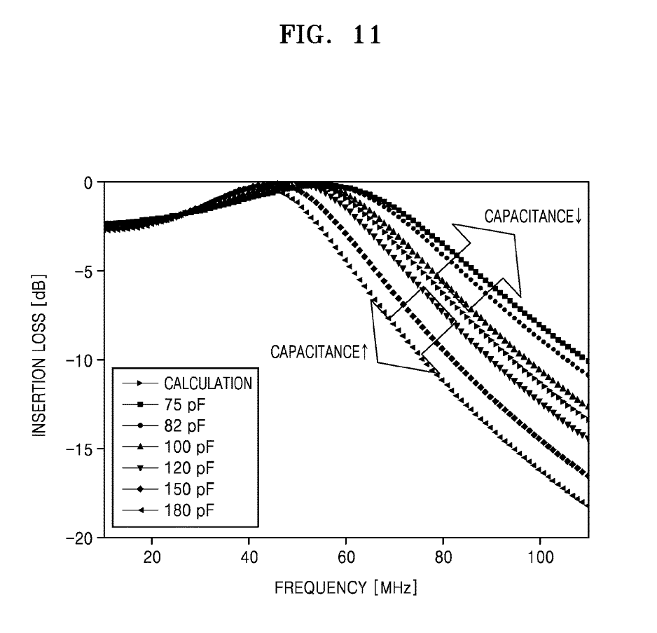

FIG. 9 is a circuit diagram a structure of an impedance matching circuit 920 operating as a low pass filter, according to an embodiment.

According to the embodiment of FIG. 9, the impedance matching circuit 920 may operate as a low-pass filter. In this case, impedance of an ultrasound probe 910 is smaller than impedance of a body 930. In order for the impedance matching circuit 920 to operate as a low-pass filter, an inductor 921 is connected to the ultrasound probe 910, and a capacitor 923 is connected to the body 930. In this case, a element value of the inductor 921 may be calculated using Equation 12, and a element value of the capacitor 923 may be calculated using Equation 11.

According to an embodiment, the impedance of the ultrasound probe 910 is equal to Equation 23, and the impedance of the body 930 is equal to Equation 24. In unique frequency characteristics of a transducer element of the ultrasound probe 910, a central frequency is 47.5 MHz, and a bandwidth is 24 MHz (35 MHz to 59 MHz).

Zₓ=21.6<−55.5°=12.236−j17.8Ω(@ 50 MHz) [Equation 23]

Zₛ50<°=50Ω(@ 50 MHz) [Equation 24]

When Equation 23 and Equation 24 are substituted into Equation 11 and Equation 12, the element value of the inductor 921 and the element value of the capacitor 923 may be calculated.

Lₓ=127.44 nH, Cₚ=107.652 pF

Characteristics of a low-pass filter with respect to a variation in the inductance of the inductor 921 from a calculated element value, and characteristics of a low-pass filter with respect to a variation in the capacitance of the capacitor 923 from a calculated element value will now be described.

FIG. 10 is a graph showing the characteristics of the impedance matching circuit 920 respectively corresponding to inductance values when the impedance matching circuit 920 operates as a low-pass filter.

According to the embodiment of FIG. 10, characteristics of a low-pass filter vary according to a variation in the element value of the inductor 921 of the impedance matching circuit 920. In detail, when the inductance of the inductor 921 increases, a cutoff frequency of the impedance matching circuit 920 operating as a low-pass filter decreases. When the inductance of the inductor 921 decreases, the cutoff frequency of the impedance matching circuit 920 operating as a low-pass filter increases. Thus, when the cutoff frequency of the impedance matching circuit 920 is desired to be changed, a controller 931 of FIG. 9 may control the inductance of the inductor 921 calculated using Equation 12 to be changed. In detail, when it is desired to decrease the cutoff frequency of the impedance matching circuit 920, the controller 931 of FIG. 9 may control the impedance matching circuit 920 to include therein an inductor having a larger inductance than the inductance of the inductor 921 calculated using Equation 12. On the other hand, when it is desired to increase the cutoff frequency of the impedance matching circuit 920, the controller 931 of FIG. 9 may control the impedance matching circuit 920 to include therein an inductor having a smaller inductance than the inductance of the inductor 921 calculated using Equation 12.

FIG. 11 is a graph showing the characteristics of the impedance matching circuit 920 respectively corresponding to capacitance values when the impedance matching circuit 920 operates as a low-pass filter.

According to the embodiment of FIG. 11, characteristics of a low-pass filter vary according to a variation in the element value of the capacitor 923 of the impedance matching circuit 920. In detail, when the capacitance of the capacitor 923 increases, a cutoff frequency of the impedance matching circuit 920 operating as a low-pass filter decreases. When the capacitance of the capacitor 923 decreases, the cutoff frequency of the impedance matching circuit 920 operating as a low-pass filter increases.

Thus, when it is desired to change the cutoff frequency of the impedance matching circuit 920, the controller 931 of FIG. 9 may change the capacitance of the capacitor 923 calculated using Equation 11. In detail, when it is desired to decrease the cutoff frequency of the impedance matching circuit 920, the controller 931 of FIG. 9 may include in the impedance matching circuit 920 a capacitor having a larger capacitance than the capacitance of the capacitor 923 calculated using Equation 11. On the other hand, when it is desired to increase the cutoff frequency of the impedance matching circuit 920, the controller 931 of FIG. 9 may include in the impedance matching circuit 920 a capacitor having a smaller capacitance than the capacitance of the capacitor 923 calculated using Equation 11.

Referring to FIGS. 10 and 11, when the impedance matching circuit 920 operates as a low-pass filter, a change in the cutoff frequency of the impedance matching circuit 920 according to the variation in the inductance value of the inductor 921, which is connected to the ultrasound probe 910, is larger than a change in the cutoff frequency of the impedance matching circuit 920 according to the variation in the capacitance value of the capacitor 923, which is connected to the body 930. Thus, when the controller 931 selects elements of the impedance matching circuit 920, the controller 931 may select the inductor 921 first and then may select the capacitor 923. When the controller 931 changes the cutoff frequency of the impedance matching circuit 920, the controller 931 may change the inductor 921 included in the impedance matching circuit 920 in preference to the capacitor 923 included in the impedance matching circuit 920.

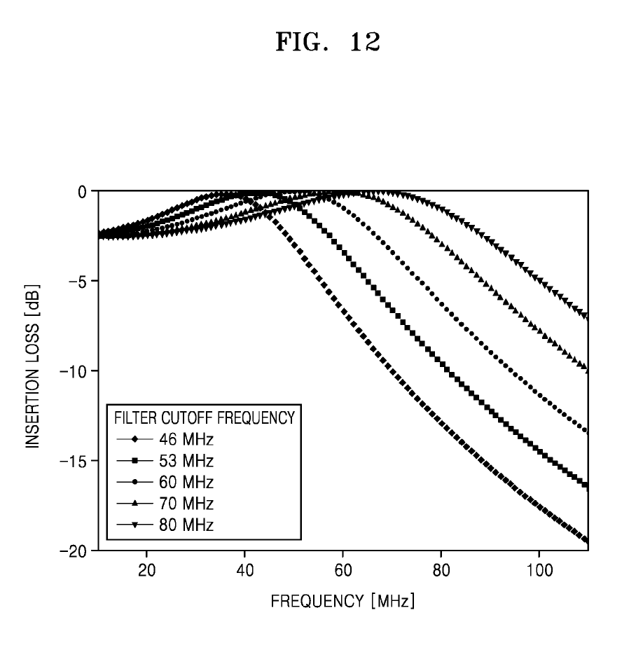

FIG. 12 is a graph showing the characteristics of the impedance matching circuit 920 respectively corresponding to cutoff frequencies when the impedance matching circuit 920 operates as a low-pass filter.

When the impedance matching circuit 920 is constructed within a unique bandwidth range (central frequency is 47.5 MHz and bandwidth is 24 MHz) of a transducer included in an ultrasound probe, the impedance matching circuit 920 has cutoff frequencies and characteristics of a low-pass filter shown in FIG. 12. Thus, the controller 931 of FIG. 9 may select circuit elements of the impedance matching circuit 920, based on the cutoff frequencies and the characteristic of the impedance matching circuit 920 operating as a low-pass filter, which shown in FIG. 12.

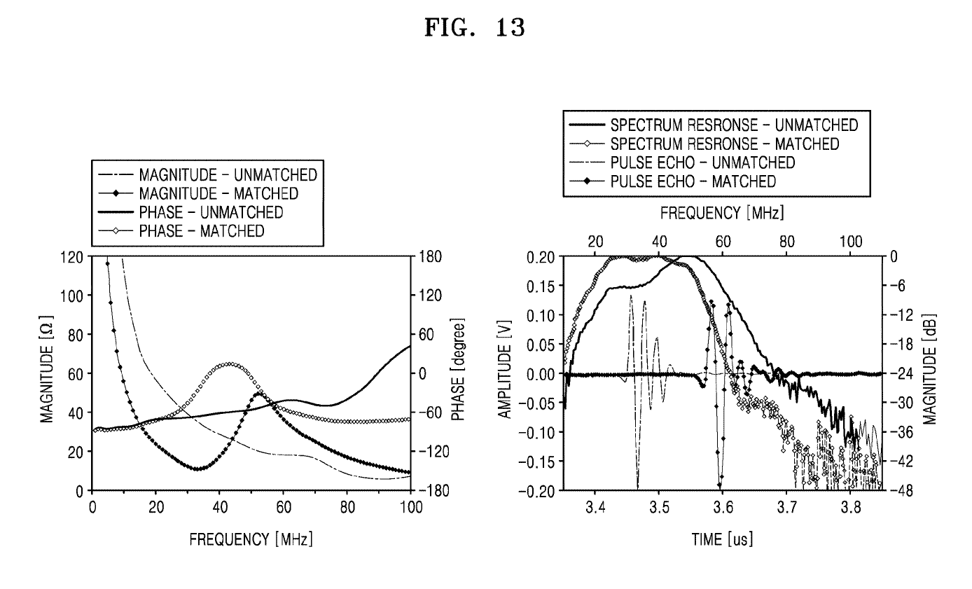

FIG. 13 is a graph of a result of impedance matching performed by using the impedance matching circuit 920 operating as a low pass filter.

FIG. 13 shows effects obtained by matching the impedance the ultrasound probe 910 with the impedance of the body 930 by using the impedance matching circuit 920 operating as a low-pass filter. The graph of FIG. 13 shows characteristics of a reception signal when the impedance matching circuit 920 exists within the unique bandwidth range (central frequency is 47.5 MHz and bandwidth is 24 MHz) of a transducer included in an ultrasound probe and characteristics of the reception signal when no impedance matching circuits exist.

When impedance matching is performed using the impedance matching circuit 920, a central frequency of a reception signal input to the body 930 is 36.1MHz, a bandwidth thereof is 32.8 MHz, an output impedance Zout thereof is 46.20, and an impedance phase thereof is −7.4°. On the other hand, when no impedance matching circuits 920 exist, a central frequency of the reception signal input to the body 930 is 47.5 MHz, a bandwidth thereof is 23.5 MHz, an output impedance Zout thereof is 21.6Ω, and an impedance phase thereof is −55.5°.

When the impedance matching circuit 920 exists, the magnitude of the bandwidth increased, the magnitude of the output impedance Zout increased, and the impedance phase decreased. As the magnitude of the output impedance Zout and the impedance magnitude of the body 930 are similar to each other, electrical energy loss of the reception signal input to the body 930 decreases. Thus, the reception signal input to the body 930 is less lost when the impedance matching circuit 920 exists than when no impedance matching circuits 920 exist.

In addition, a signal response of a 25 MHz to 45 MHz band is better when the impedance matching circuit 920 is used than when the impedance matching circuit 920 is not used. Thus, loss of the reception signal input to the body 930 is reduced within a unique bandwidth of a transducer included in the ultrasound probe 910 when the impedance matching circuit 920 is used, compared to when no impedance matching circuits 920 are used.

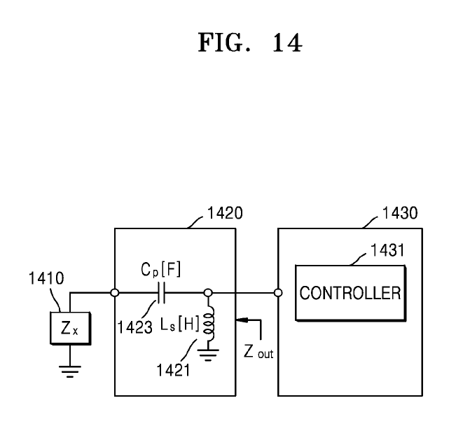

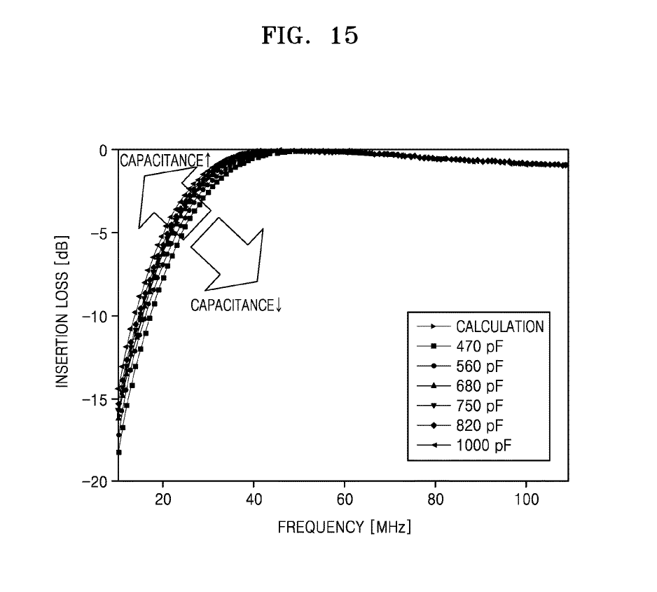

FIG. 14 is a circuit diagram a structure of an impedance matching circuit 1420 operating as a high pass filter, according to an embodiment.

According to the embodiment of FIG. 14, the impedance matching circuit 1420 may operate as a high-pass filter. In this case, impedance of an ultrasound probe 1410 is smaller than impedance of a body 1430. In order for the impedance matching circuit 1420 to operate as a high-pass filter, a capacitor 1423 is connected to the ultrasound probe 1410, and an inductor 1421 is connected to the body 1430. In this case, a element value of the inductor 1421 may be calculated using Equation 11, and a element value of the capacitor 1423 may be calculated using Equation 12.

According to an embodiment, the impedance of the ultrasound probe 1410 is equal to Equation 23, and the impedance of the body 1430 is equal to Equation 24. In unique frequency characteristics of a transducer element of the ultrasound probe 1410, a central frequency is 47.5 MHz, and a bandwidth is 24 MHz (35 MHz to 59 MHz).

When Equation 23 and Equation 24 are substituted into Equation 11 and Equation 12, the element value of the inductor 1421 and the element value of the capacitor 1423 may be calculated.

C_{S}=702.303 pF, L_{P}=94.119 nH

Characteristics of a high-pass filter with respect to a variation in the inductance of the inductor 1421 from a calculated element value, and characteristics of a high-pass filter with respect to a variation in the capacitance of the capacitor 1423 from a calculated element value will now be described.

FIG. 15 is a graph showing the characteristics of the impedance matching circuit 1420 respectively corresponding to capacitance values when the impedance matching circuit 1420 operates as a high-pass filter.

According to the embodiment of FIG. 15, characteristics of a high-pass filter vary according to a variation in the element value of the capacitor 1423 of the impedance matching circuit 1420. In detail, when the capacitance of the capacitor 1423 increases, a cutoff frequency of the impedance matching circuit 1420 operating as a high-pass filter decreases. When the capacitance of the capacitor 1423 decreases, the cutoff frequency of the impedance matching circuit 1420 operating as a high-pass filter increases.

Thus, when the cutoff frequency of the impedance matching circuit 1420 is desired to be changed, the controller 1431 of FIG. 14 may change the capacitance of the capacitor 1423 calculated using Equation 12. In detail, when it is desired to decrease the cutoff frequency of the impedance matching circuit 1420, the controller 1431 may include in the impedance matching circuit 1420 a capacitor having a larger capacitance than the capacitance of the capacitor 1423 calculated using Equation 12. On the other hand, when it is desired to increase the cutoff frequency of the impedance matching circuit 1420, the controller 1431 may control the impedance matching circuit 1420 to include therein a capacitor having a smaller capacitance than the capacitance of the capacitor 1423 calculated using Equation 12.

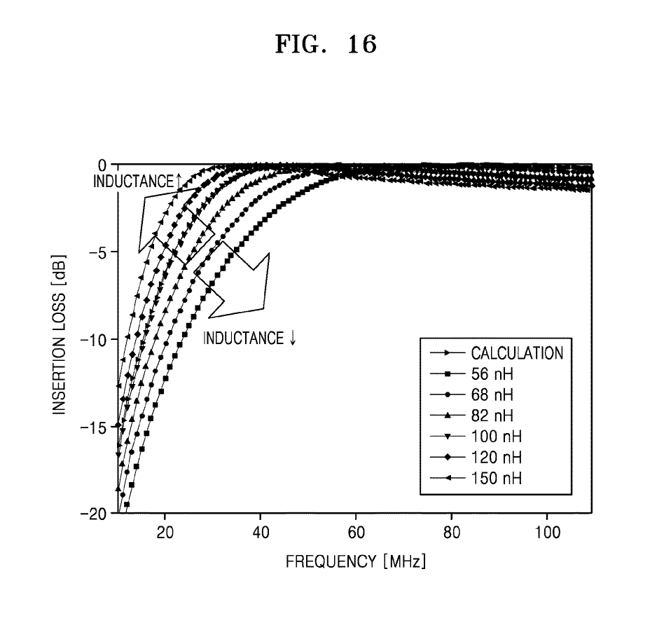

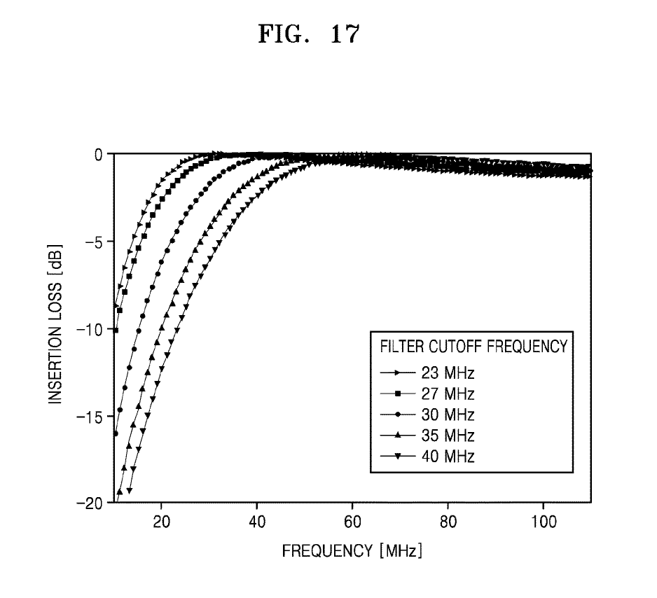

FIG. 16 is a graph showing the characteristics of the impedance matching circuit 1420 respectively corresponding to inductance values when the impedance matching circuit 1420 operates as a high-pass filter.

According to the embodiment of FIG. 16, characteristics of a high-pass filter vary according to a variation in the element value of the inductor 1421 of the impedance matching circuit 1420. In detail, when the inductance of the inductor 1421 increases, a cutoff frequency of the impedance matching circuit 1420 operating as a high-pass filter decreases. When the inductance of the inductor 1421 decreases, the cutoff frequency of the impedance matching circuit 1420 operating as a high-pass filter increases.

Thus, when the cutoff frequency of the impedance matching circuit 1420 is desired to be changed, the controller 1431 of FIG. 14 may change the inductance of the inductor 1421 calculated using Equation 11. In detail, when it is desired to decrease the cutoff frequency of the impedance matching circuit 1420, the controller 1431 may include in the impedance matching circuit 1420 an inductor having a larger inductance than the inductance of the inductor 1421 calculated using Equation 11. On the other hand, when it is desired to increase the cutoff frequency of the impedance matching circuit 1420, the controller 1431 may include in the impedance matching circuit 1420 an inductor having a smaller inductance than the inductance of the inductor 1421 calculated using Equation 11.

Referring to FIGS. 15 and 16, when the impedance matching circuit 1420 operates as a high-pass filter, a change in the cutoff frequency of the impedance matching circuit 1420 according to the variation in the inductance value of the inductor 1421, which is connected to the body 1430, is larger than a change in the cutoff frequency of the impedance matching circuit 1420 according to the variation in the capacitance value of the capacitor 1423, which is connected to the ultrasound probe 1410. Thus, when the controller 1431 selects circuit elements included in the impedance matching circuit 1420, the controller 1431 may select the inductor 1421 first and then may select the capacitor 1423. When the controller 1431 changes the cutoff frequency of the impedance matching circuit 1420, the controller 1431 may change the inductor 1421 included in the impedance matching circuit 1420 in preference to the capacitor 1423 included in the impedance matching circuit 1420.

FIG. 17 is a graph showing the characteristics of the impedance matching circuit 1420 respectively corresponding to cutoff frequencies when the impedance matching circuit 1420 operates as a high-pass filter.

When the impedance matching circuit 1420 is constructed within a unique bandwidth range (central frequency is 47.5 MHz and bandwidth is 24 MHz) of a transducer included in an ultrasound probe, the impedance matching circuit 1420 has cutoff frequencies and characteristics of a high-pass filter shown in FIG. 17. Thus, the controller 1431 may select circuit elements included in the impedance matching circuit 1420, based on the cutoff frequencies and the characteristic of the impedance matching circuit 1420 operating as a high-pass filter, which are shown in FIG. 17.

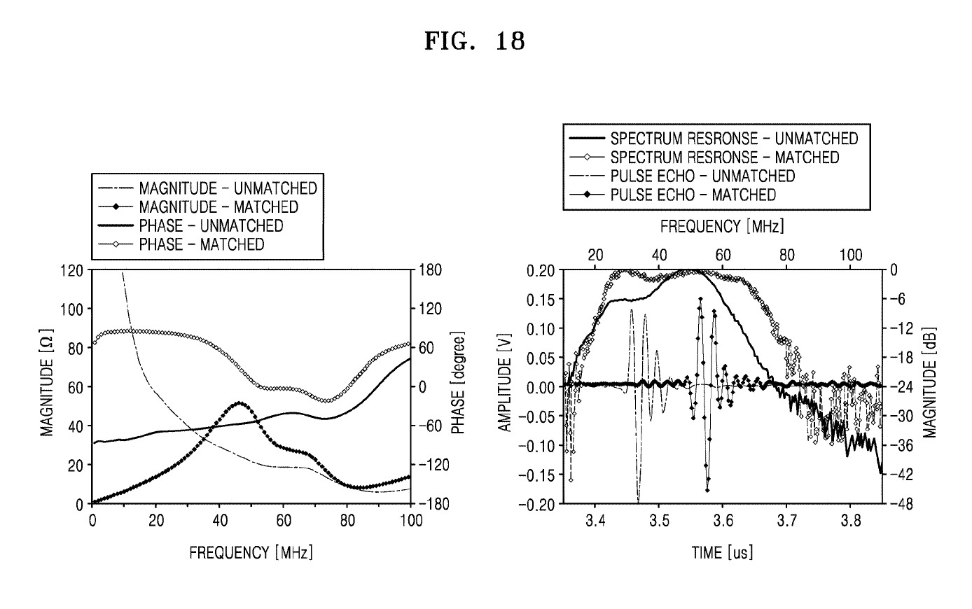

FIG. 18 is a graph showing a result of impedance matching performed by using the impedance matching circuit 1420 operating as a high pass filter.

FIG. 18 shows effects obtained by matching the impedance the ultrasound probe 1410 with the impedance of the body 1430 by using the impedance matching circuit 1420 operating as a high-pass filter. The graph of FIG. 18 shows characteristics of a reception signal when the impedance matching circuit 1420 exists within the unique bandwidth range (central frequency is 47.5 MHz and bandwidth is 24 MHz) of a transducer included in an ultrasound probe and characteristics of the reception signal when no impedance matching circuits exist.

When impedance matching is performed using the impedance matching circuit 1420, a central frequency of a reception signal input to the body 1430 is 46.8 MHz, a bandwidth thereof is 47.5 MHz, an output impedance Zout thereof is 46.8Ω, and an impedance phase thereof is −8.9°. On the other hand, when no impedance matching circuits 1420 exist, a central frequency of the reception signal input to the body 1430 is 47.5 MHz, a bandwidth thereof is 23.5 MHz, an output impedance Zout thereof is 21.6.Ω, and an impedance phase thereof is −55.5 °.

When the impedance matching circuit 1420 exists, the bandwidth increased, the output impedance Zout increased, and the impedance phase decreased. As the magnitude of the output impedance Zout and the impedance magnitude of the body 1430 are similar to each other, electrical energy loss of the reception signal input to the body 1430 decreases. Thus, the reception signal input to the body 1430 is less lost when the impedance matching circuit 1420 exists than when no impedance matching circuits 1420 exist.

In addition, a signal response of a 25 MHz to 50 MHz band is better when the impedance matching circuit 1420 is used than when the impedance matching circuit 1420 is not used. Thus, loss of the reception signal input to the body 1430 is reduced within a unique bandwidth of a transducer included in the ultrasound probe 1410 when the impedance matching circuit 1420 is used, compared to when no impedance matching circuits 1420 are used.

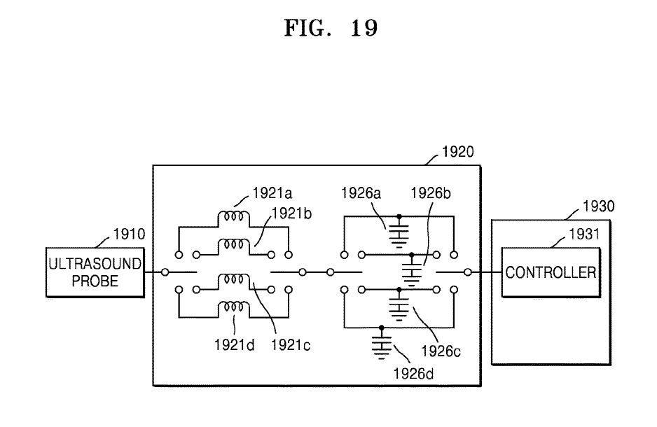

FIG. 19 is a block diagram of a structure of an ultrasound diagnosis apparatus including an impedance matching circuit 1920 including a plurality of circuit elements of an identical type having different values, according to an embodiment.

According to the embodiment of FIG. 19, the impedance matching circuit 1920 may include the plurality of circuit elements of an identical type having different values. Although the plurality of circuit elements are a serial inductor 1921 and a parallel capacitor 1926 in FIG. 19, embodiments are not limited thereto. The impedance matching circuit 1920 may include a serial capacitor and a parallel inductor. Although an impedance magnitude of an ultrasound probe 1910 is smaller than that of a body 1930 in FIG. 19, embodiments are not limited thereto.

According to the embodiment of FIG. 19, the inductance value of serial inductors 1921a, 1921b, 1921c, and 1921d may be 82 nH, 100 nH, 120 nH, and 150 nH, respectively, and the capacitance values of parallel capacitors 1926a, 1926b, 1926c, and 1926d may be 82 pF, 100 pF, 120 pF, and 150 pF, respectively. A controller 1931 may select one from the plurality of serial inductors 1921a, 1921b, 1921c, and 1921d and one from the parallel capacitors 1926a, 1926b, 1926c, and 1926d, by using element values determined according to impedance of the ultrasound probe 1910, impedance of the body 1930, and relevant frequency characteristics of a reception signal. The controller 1931 may control the impedance matching circuit 1920 to operate as a filter by using the selected serial inductor and the selected parallel capacitor.

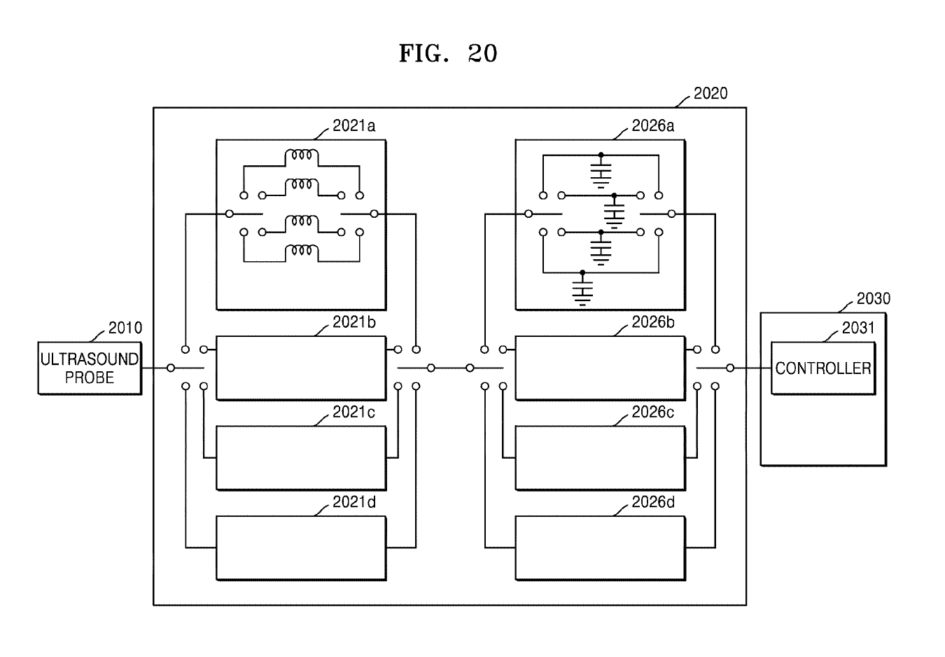

FIG. 20 is a circuit diagram of a structure of an ultrasound diagnosis apparatus including an impedance matching circuit 2020 including a plurality of circuit element banks, according to an embodiment.

According to the embodiment of FIG. 20, the impedance matching circuit 2020 may include a plurality of circuit element banks 2021a, 2021b, 2021c, 2021d, 2026a, 2027b, 2028c, and 2029d. Each of the plurality of circuit element banks 2021a, 2021b, 2021c, 2021d, 2026a, 2027b, 2028c, and 2029d may include a plurality of circuit elements of different types having different values. The plurality of circuit elements may be variable elements of which element values are variable. The circuit elements included in each of the plurality of circuit element banks may be circuit elements of different types. For example, a plurality of circuit elements (for example, serial inductors) included in a first circuit element bank 2021amay be of different types from a plurality of circuit elements (for example, parallel capacitors) included in a second circuit element bank 2026a.

According to an embodiment, the circuit element bank 2021afrom among the plurality of circuit element banks may include serial inductors having different values. The circuit element bank 2026a from among the plurality of circuit element banks may include parallel capacitors having different values. The circuit element bank 2021b from among the plurality of circuit element banks may include serial capacitors having different values. The circuit element bank 2021c from among the plurality of circuit element banks may include parallel inductors having different values. The circuit element bank 2021d from among the plurality of circuit element banks may include parallel capacitors having different values. The circuit element bank 2026b from among the plurality of circuit element banks may include parallel inductors having different values. The circuit element bank 2026c from among the plurality of circuit element banks may include serial inductors having different values. The circuit element bank 2026d from among the plurality of circuit element banks may include serial capacitors having different values.

To select impedance of an ultrasound probe 2010 with impedance of a body 2030, a controller 2031 may select one from the plurality of circuit element banks included in the impedance matching circuit 2020 and may select one from the plurality of circuit elements included in the selected circuit element bank.

The controller 2031 may control the impedance matching circuit 2020 to match the impedance of the ultrasound probe 2010 with the impedance of the body 2030 by using the selected circuit element. The controller 2031 may control switches included in the impedance matching circuit 2020 to connect the impedance matching circuit 2020 to the selected circuit element.

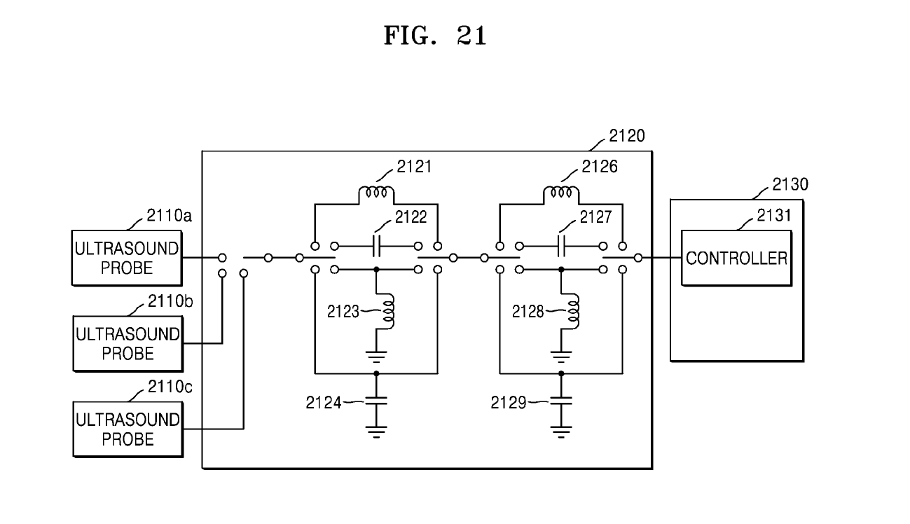

FIG. 21 is a block diagram of a structure of an ultrasound diagnosis apparatus including an impedance matching circuit 2120 to which a plurality of ultrasound probes is connected, according to an embodiment.

According to the embodiment of FIG. 21, the ultrasound diagnosis apparatus 100 may include a plurality of ultrasound probes 2110a, 2110b, and 2110c, one of which may be connected to a body 2130. The plurality of ultrasound probes 2110a, 2110b, and 2110c may be ultrasound probes of an identical type or may be ultrasound probes of different types.

A controller 2131 may detect an ultrasound probe currently-being-used by a user from among the plurality of ultrasound probes 2110a, 2110b, and 2110c. The controller 2131 may control the impedance matching circuit 2120, based on the type and application of the ultrasound probe currently-being-used by the user.

According to an embodiment, at least one of the first ultrasound probe 2110a including a single transducer element and having a smaller impedance magnitude than the body 2130, a second ultrasound probe 2110b including a transducer array of a plurality of transducer elements and having a larger impedance magnitude than the body 2130, and a third ultrasound probe 2110c including a single transducer element and having a larger impedance magnitude than the body 2130 may be connected to the body 2130.

According to an embodiment, when a user uses the first ultrasound probe 2110a, the controller 2131 may detect the first ultrasound probe 2110a from the at least one ultrasound probe connected to the body 2130. The controller 2131 may select circuit elements that are included in the impedance matching circuit 2120 to correspond to the detected first ultrasound probe 2110a. In this case, the controller 2131 may select a serial inductor 2121 and a parallel capacitor 2129.

According to an embodiment, when the user uses the second ultrasound probe 2110b, the controller 2131 may detect the second ultrasound probe 2110b from the at least one ultrasound probe connected to the body 2130 and may select circuit elements of the impedance matching circuit 2120 corresponding to the detected second ultrasound probe 2110b. In this case, the controller 2131 may select a parallel capacitor 2124 and a serial inductor 2126.

According to an embodiment, a storage may store identification information for identifying the types of ultrasound probes. The controller 2131 may use the identification information stored in the storage, to identify the type of ultrasound probe connected to the body 2130. The controller 2131 may extract identification information for identifying the type of ultrasound probe, from the ultrasound probe connected to the body 2130. The controller 2131 may compare the identification information stored in the storage with the identification information extracted from the ultrasound probe. The controller 2131 may select circuit elements of the impedance matching circuit 2120 by using a result of the comparison of the identification information, and may control the impedance matching circuit 2120 to match impedance of the ultrasound probe connected to the body 2130 with impedance of the body 2130 by using the selected circuit elements. In this case, the storage may store identification information, information about a circuit element corresponding to the identification information, information about circuit elements respectively corresponding to a plurality of cutoff frequencies, information about a circuit element corresponding to relevant frequency characteristics, and information about relevant frequency characteristics respectively corresponding to a plurality of applications. The storage may arrange the stored various pieces of information into a table and store the table. The controller 2131 may construct the impedance matching circuit 2120 by using the various pieces of information stored in the storage. The controller 2131 may update the various pieces of information stored in the storage.

According to an embodiment, the storage may store information about the types of ultrasound probes. A user input interface may receive a user input of selecting one from the types of ultrasound probes stored in the storage. The controller 2131 may detect, from the storage, information about circuit elements of the impedance matching circuit 2120 corresponding to the ultrasound probe selected based on the user input. The controller 2131 may control the impedance matching circuit 2120 to be constructed using the information about the circuit elements detected from the storage, and may control the impedance of ultrasound probes 2110a, 2110b, and 2110c with the impedance of the body 2130.

According to an embodiment, when identification information detected from the ultrasound probes 2110a, 2110b, and 2110c is different from the identification information stored in the storage, the controller 2131 may control measurement of the impedance of the ultrasound probe 21s 2110a, 2110b, and 2110c 10. The controller 2131 may control measurement of the impedance of the body 2130. The controller 2131 may select circuit elements that are included in the impedance matching circuit 2120 so that the impedance of the ultrasound probes 2110a, 2110b, and 2110c is matched with the impedance of the body 2130 by using the measured impedance of the ultrasound probes 2110a, 2110b, and 2110c. The controller 2131 may control the impedance matching circuit 2120 to match the impedance of the ultrasound probes 2110a, 2110b, and 2110c with the impedance of the body 2130 by using the selected circuit elements.

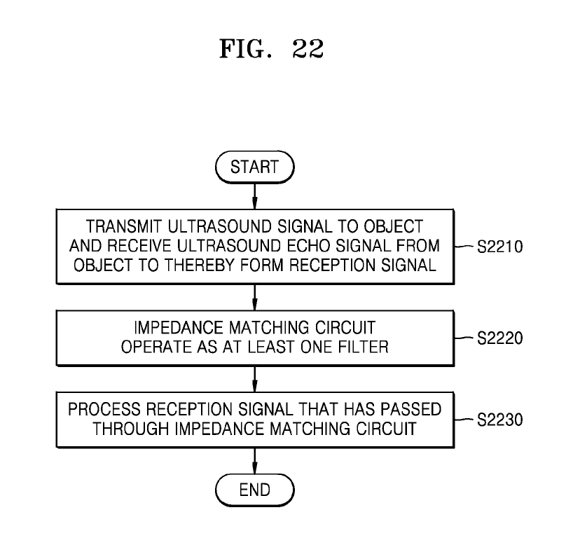

FIG. 22 is a flowchart of a method of controlling the ultrasound diagnosis apparatus 100, according to an embodiment.

According to the embodiment of FIG. 22, the ultrasound diagnosis apparatus 100 may transmit an ultrasound signal to an object and receive an ultrasound echo signal from the object to thereby form a reception signal, in operation S2210. The impedance matching circuit 320 may operate as at least one filter, in operation S2220. The ultrasound diagnosis apparatus 100 may process a reception signal that has passed through the impedance matching circuit 320, in operation S2230.

In detail, in operation S2210, the ultrasound diagnosis apparatus 100 may transmit an ultrasound signal to an object by using the ultrasound probe 310 and receive an ultrasound echo signal from the object to thereby form a reception signal. In this case, the ultrasound diagnosis apparatus 100 may differently set the frequencies of ultrasound signals transmitted to different objects, according to the type of ultrasound probe 310, an application of the ultrasound probe 310, and the type of object that is to be diagnosed.

In operation S2220, the impedance matching circuit 320 may operate as a filter that stops or passes a specific frequency of the reception signal. This operation has already described above in detail.

In operation S2220, the impedance matching circuit 320 may operate as a filter so that the reception signal that has passed through the impedance matching circuit 320 includes changed relevant frequency characteristics based on a received user input.

In operation S2230, the ultrasound diagnosis apparatus 100 processes the reception signal that has passed through the impedance matching circuit 320. According to an embodiment, the ultrasound diagnosis apparatus 100 may generate an ultrasound image by using the reception signal and display the ultrasound image. The ultrasound diagnosis apparatus 100 may use a well-known method of processing an ultrasound reception signal.

According to an embodiment, the ultrasound diagnosis apparatus 100 may receive a user input of changing relevant frequency characteristics. In this case, the user input of changing relevant frequency characteristics may include an input of changing the application of the ultrasound probe, an input of selecting an application, an input of changing the cutoff frequency of a filter, and an input of selecting one from a plurality of ultrasound probes connected to a body.

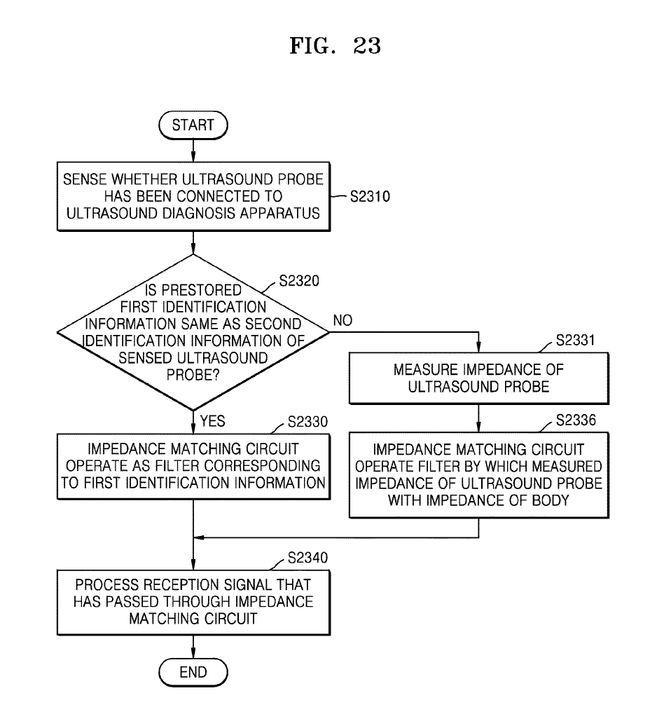

FIG. 23 is a flowchart of a method of controlling the ultrasound diagnosis apparatus 100, according to an embodiment.

According to the embodiment of FIG. 23, the ultrasound diagnosis apparatus 100 may sense whether the ultrasound probe 310 has been connected to the ultrasound diagnosis apparatus 100, in operation S2310. In operation S2320, to detect the application and the type of the connected ultrasound probe 310, the ultrasound diagnosis apparatus 100 may compare first identification information stored in the storage 150 with second identification information extracted from the sensed ultrasound probe 310. In operation S2330, when the first identification information is the same as the second identification information, the impedance matching circuit 320 may operate as a filter corresponding to the first identification information. In operation S2331, when the first identification information is not the same as the second identification information, the ultrasound diagnosis apparatus 100 may measure impedance of the ultrasound probe 310. In operation S2336, the impedance matching circuit 320 that matches the measured impedance with the impedance of the body 330 may operate as a filter. In operation S2340, when impedance matching circuit 320 operates as a filter, the ultrasound diagnosis apparatus 100 may process a reception signal that has passed through the impedance matching circuit 320.

In operation S2330, the ultrasound diagnosis apparatus 100 may select circuit elements of the impedance matching circuit 320 by using information about elements of the impedance matching circuit 320 corresponding to the first identification information stored in the storage 150. The storage 150 may store identification information, information about a circuit element corresponding to the identification information, information about circuit elements respectively corresponding to a plurality of cutoff frequencies, information about a circuit element corresponding to relevant frequency characteristics, and information about relevant frequency characteristics respectively corresponding to a plurality of applications. The storage 150 may arrange the stored various pieces of information into a table and store the table. The information about elements of the impedance matching circuit 320 corresponding to the first identification information may include the types of circuit elements, element values of the circuit elements, and a method of connecting the circuit elements.

In operation S2331, the ultrasound diagnosis apparatus 100 may receive a user input regarding the application and type of an ultrasound probe. The ultrasound diagnosis apparatus 100 may determine the application and type of an ultrasound probe, based on the received user input. The ultrasound diagnosis apparatus 100 may control the impedance matching circuit 320 to operate as a filter corresponding to the determined application and type of the ultrasound probe.

The above-described embodiments of the present inventive concept may be embodied in form of a computer-readable recording medium for storing computer executable command languages and data. The command languages may be stored in form of program codes and, when executed by a processor, may perform a certain operation by generating a certain program module. Also, when executed by a processor, the command languages may perform certain operations of the disclosed embodiments.

What is claimed is:

1. An ultrasound diagnosis apparatus comprising:
an ultrasound probe configured to transmit an ultrasound signal to an object and receive an ultrasound echo signal from the object to form a reception signal;
a body configured to receive the reception signal from the ultrasound probe and process the reception signal;
an impedance matching circuit configured to match an impedance of the ultrasound probe with an impedance of the body; and
a controller configured to control the impedance matching circuit to operate as at least one filter that passes or rejects a signal of a specific frequency band, based on the impedance of the ultrasound probe, the impedance of the body, and relevant frequency characteristics of the reception signal,
wherein the at least one filter is controlled to match the impedance of the ultrasound probe with the impedance of the body and is controlled so that the reception signal has the relevant frequency characteristics.

2. The ultrasound diagnosis apparatus of claim 1, wherein the controller is configured to control the impedance matching circuit to operate as a multi-stage filter.

3. The ultrasound diagnosis apparatus of claim 1, wherein
the impedance matching circuit comprises a plurality of circuit elements of an identical type having different values, and
the controller is configured to control the impedance matching circuit to match the impedance of the ultrasound probe with the impedance of the body by using at least one of the plurality of circuit elements.

4. The ultrasound diagnosis apparatus of claim 1, wherein
the impedance matching circuit comprises a plurality of circuit elements of different types, and
the controller is configured to control the impedance matching circuit to match the impedance of the ultrasound probe with the impedance of the body by using at least one of the plurality of circuit elements.

5. The ultrasound diagnosis apparatus of claim 1, wherein
the impedance matching circuit comprises a plurality of circuit element banks of different types,
each of the plurality of circuit element banks comprises a plurality of circuit elements of an identical type having different values, and
the controller is configured to control the impedance matching circuit to match the impedance of the ultrasound probe with the impedance of the body by using at least one of the plurality of circuit elements.

6. The ultrasound diagnosis apparatus of claim 1, wherein
the impedance matching circuit comprises a plurality of circuit elements of different types,
the plurality of circuit elements are variable elements of which values are variable, and
the controller is configured to control a value of at least one of the plurality of circuit elements, based on the impedance of the ultrasound probe and the impedance of the body.

7. The ultrasound diagnosis apparatus of claim 1, further comprising an input interface configured to receive from a user an input for changing the relevant frequency characteristics,
wherein the controller is configured to control the impedance matching circuit to operate as a filter corresponding to changed relevant frequency characteristics.

8. The ultrasound diagnosis apparatus of claim 1, wherein
a plurality of ultrasound probes are included, and are connected to the body, and
the controller is configured to detect a first probe that receives the ultrasound echo signal, from among the plurality of ultrasound probes, and to control the impedance matching circuit to match impedance of the first probe with the impedance of the body.

9. The ultrasound diagnosis apparatus of claim 1, further comprising:
a storage configured to store a plurality of applications used for ultrasound diagnosis; and
an input interface configured to receive, from a user, an input for selecting one from the plurality of stored applications,
wherein the controller is configured to run the selected application in response to the input received by the input interface, and to control the impedance matching circuit so that the reception signal has the relevant frequency characteristics corresponding to the selected application.

10. The ultrasound diagnosis apparatus of claim 1, further comprising:
a storage configured to store information about a type of the ultrasound probe; and
an input interface configured to receive, from a user, an input for selecting one from the stored information about the type of the ultrasound probe,
wherein the controller is configured to control the impedance matching circuit to operate as a filter corresponding to the selected information about the type of the ultrasound probe.

11. The ultrasound diagnosis apparatus of claim 1, further comprising a storage configured to store first identification information for identifying a type of the ultrasound probe,
wherein the controller is configured to detect second identification information from the ultrasound probe connected to the body, to compare the first identification information with second identification information, and to control the impedance matching circuit according to a result of the comparing of the first identification information with the second identification information.

12. The ultrasound diagnosis apparatus of claim 11, wherein, when the first identification information is the same as the second identification information, the controller is configured to control the impedance matching circuit to operate as a filter corresponding to the first identification information.

13. The ultrasound diagnosis apparatus of claim 11, wherein, when the first identification information is not the same as the second identification information, the controller is configured to control measurements of the impedance of the ultrasound probe and the impedance of the body and to control the impedance matching circuit to match the measured impedance of the ultrasound probe with the measured impedance of the body.

14. A method of controlling an ultrasound diagnosis apparatus comprising an ultrasound probe and a body for processing a reception signal received from the ultrasound probe, the method comprising:
transmitting an ultrasound signal to an object and receiving an ultrasound echo signal from the object to form the reception signal;
controlling an impedance matching circuit for matching an impedance of the ultrasound probe with an impedance of the body to operate as at least one filter that passes or rejects a signal of a specific frequency band, based on the impedance of the ultrasound probe, the impedance of the body, and relevant frequency characteristics of the reception signal; and
processing a reception signal that has passed through the impedance matching circuit,
wherein the reception signal that has passed through the impedance matching circuit has the relevant frequency characteristics.

15. The method of claim 14, further comprising sensing the ultrasound probe connected to the body,
wherein the controlling of the impedance matching circuit to operate as the at least one filter that passes or rejects the signal of the specific frequency band comprises controlling the impedance matching circuit to operate as a filter corresponding to a type of the sensed ultrasound probe.

16. The method of claim 15, wherein the sensing of the ultrasound probe connected to the body comprises:
comparing first identification information stored in a storage with second identification information of the ultrasound probe; and
determining the type of the sensed ultrasound probe based on a result of the comparing of the first identification information with the second identification information.

17. The method of claim 16, wherein, when the first identification information is the same as the second identification information, the controlling of the impedance matching circuit to operate as the filter corresponding to the type of the sensed ultrasound probe comprises controlling the impedance matching circuit to operate as a filter corresponding to the identification information.

18. The method of claim 15, wherein, when the first identification information is not the same as the second identification information, the controlling of the impedance matching circuit to operate as the at least one filter that passes or rejects the signal of the specific frequency band comprises:
measuring the impedance of the ultrasound probe; and
controlling the impedance matching circuit to operate as a filter so that the measured impedance of the ultrasound probe is matched with the impedance of the body.

19. The method of claim 14, further comprising receiving from a user an input for changing the relevant frequency characteristics,
wherein the controlling of the impedance matching circuit to operate as the at least one filter that passes or rejects the signal of the specific frequency band comprises controlling the impedance matching circuit to operate as a filter so that the reception signal that has passed through the impedance matching circuit has the changed relevant frequency characteristics.

20. The method of claim 19, wherein the input for changing the relevant frequency characteristics comprises at least one of an input for changing an application of the ultrasound probe, an input for selecting an application, an input for changing a cutoff frequency of the at least one filter, and an input for selecting one from a plurality of ultrasound probes connected to the body.
